SEQUENCE LISTING (27 sequences)

SEQ ID NO: 1 - protein, 15 aa, Artificial Sequence
  Synthetic peptide
EDYSXXTSNPIAEYK

SEQ ID NO: 2 - protein, 8 aa, Artificial Sequence
  Synthetic peptide
IQIGDSLV

SEQ ID NO: 3 - protein, 11 aa, Artificial Sequence
  Synthetic peptide
STASLGVTLSV

SEQ ID NO: 4 - protein, 13 aa, Artificial Sequence
  Synthetic peptide
AGTLRPEKPFTAN

SEQ ID NO: 5 - protein, 16 aa, Artificial Sequence
  Synthetic peptide
DGWVVSPSENYNHWAE

SEQ ID NO: 6 - protein, 9 aa, Artificial Sequence
  Synthetic peptide
IQVXDEVXE

SEQ ID NO: 7 - protein, 13 aa, Artificial Sequence
  Synthetic peptide
YAGDTITGNTDNS

SEQ ID NO: 8 - DNA, 20 bp, Artificial Sequence
  Synthetic primer
aayccnatngcngartayaa

SEQ ID NO: 9 - DNA, 20 bp, Artificial Sequence
  Synthetic primer
aaytayaaycattgggcnga

SEQ ID NO: 10 - DNA, 23 bp, Artificial Sequence
  Synthetic primer
ccncaraarccnttyacngcnaa

SEQ ID NO: 11 - DNA, 84 bp, Artificial Sequence
  GhPRP1 partial nucleotide sequence
ccccagaagccatttactgcgaacaagcttccgtttattctctacaccgttgggccatttgctttcgaacccaaatgctactag

SEQ ID NO: 12 - protein, 27 aa, Artificial Sequence
  GhPRP1 partial amino acid sequence
PEKPFTANKLPFILYTVGPFAFEPKCY

SEQ ID NO: 13 - DNA, 22 bp, Artificial Sequence
  Synthetic primer
gctatttctatagcaactcaac

SEQ ID NO: 14 - DNA, 24 bp, Artificial Sequence
  Synthetic primer
caaactcaaaacaaccccaaaacc

SEQ ID NO: 15 - DNA, 22 bp, Artificial Sequence
  Synthetic primer
gatgaaagcaaggcacacacac

SEQ ID NO: 16 - DNA, 20 bp, Artificial Sequence
  Synthetic primer
ccccttaataattcagcacc

SEQ ID NO: 17 - DNA, 528 bp, Gossypium sp.
atggctgctaaagctttttcaagaagtataactcctttggtgcttttgttcatatttttaagctttgcacaaggtaaagaaatcatggttggtggcaaaacaggcgcttggaagataccttcttctgaatcagattctctcaacaaatgggctgaaaaagctcgtttccaaatcggcgactctctcgtgtggaaatatgatggtggtaaagactcggtgctccaagtgagtaaggaggattatacaagttgcaatacgtcgaacccgattgccgagtacaaagatgggaacaccaaggtgaagcttgaaaagtcaggaccatatttcttcatgagtggagcaaagggccactgcgagcaaggccagaagatgattgtggttgtgatgtctcaaaagcataggtacattggaatctctccagcaccttcgccggttgattttgaaggtccggccgttgctccaacaagcggagttgcagggttgaaggctggtttgttggtgacagtgggggttttggggttgttttga

SEQ ID NO: 18 - protein, 175 aa, Gossypium sp.
MAAKAFSRSITPLVLLFIFLSFAQGKEIMVGGKTGAWKIPSSESDSLNKWAEKARFQIGDSLVWKYDGGKDSVLQVSKEDYTSCNTSNPIAEYKDGNTKVKLEKSGPYFFMSGAKGHCEQGQKMIVVVMSQKHRYIGISPAPSPVDFEGPAVAPTSGVAGLKAGLLVTVGVLGLF

SEQ ID NO: 19 - DNA, 660 bp, Gossypium sp.
atggggttcgaaaggtatcttgctagtgtgttgatagtgataatgctgtcttttatcacttcatcacagggttataagttctatgttggtggtagagacggttgggttgttagtccttctgagaactacaatcattgggctgaaaggaatagattccaagtcaatgatactctctttttcaagtacaagaaagggtcagactcggtgctgttggtaacaagagaagattacttctcatgcaacaccaagaacccaattcagtctttaacagaaggtgattcactctttacatttgatcggtcgggtcccttctttttcatcaccggtaacgctgataattgcaaaaaagggcaaaagctgatcgtcgtggtcatggctgtaagacacaaaccccagcaacaacctccttcaccttctccctcatctgctgtgacaacagcgccggtttctccacccacattacccattcctgaaactaaccctcctgtagagtcaccaaagagcagtgaggctccatctcatgatgctgtggaaccagctccgccggagcacagatcgggttcattcaaactagtatgttctacctggctggtgttgggtttcggcatttgggtcagcatggccttggggatcgaaaatgtagtttgtttttggtgctga

SEQ ID NO: 20 - protein, 219 aa, Gossypium sp.
MGFERYLASVLIVIMLSFITSSQGYKFYVGGRDGWVVSPSENYNHWAERNRFQVNDTLFFKYKKGSDSVLLVTREDYFSCNTKNPIQSLTEGDSLFTFDRSGPFFFITGNADNCKKGQKLIVVVMAVRHKPQQQPPSPSPSSAVTTAPVSPPTLPIPETNPPVESPKSSEAPSHDAVEPAPPEHRSGSFKLVCSTWLVLGFGIWVSMALGIENVVCFWC

SEQ ID NO: 21 - DNA, 48 bp, Artificial Sequence
  Synthetic primer
caccctggttccgcgtggatccaaagaaatcatggttggtggcaaaac

SEQ ID NO: 22 - DNA, 31 bp, Artificial Sequence
  Synthetic primer
ctagattccaatgtacctatgcttttgagac

SEQ ID NO: 23 - DNA, 45 bp, Artificial Sequence
  Synthetic primer
caccctggttccgcgtggatcctataagttctatgttggtggtag

SEQ ID NO: 24 - DNA, 34 bp, Artificial Sequence
  Synthetic primer
ctattgttgctggggtttgtgtcttacagccatg

SEQ ID NO: 25 - protein, 147 aa, Artificial Sequence
  Recombinant PL1 sequence
MSYYHHHHHHLESTSLYKKAGSAAAPFTLVPRGSKEIMVGGKTGAWKIPSSESDSLNKWAEKARFQIGDSLVWKYDGGKDSVLQVSKEDYTSCNTSNPIAEYKDGNTKVKLEKSGPYFFMSGAKGHCEQGRKMIVVVMSQKHRYIGI

SEQ ID NO: 26 - protein, 144 aa, Artificial Sequence
  Recombinant P12 sequence
MSYYHHHHHHLESTSLYKKAGSAAAPFTLVPRGSYKFYVGGRDGWVVSPSENYNHWAERNRFQVNDTLFFKYKKGSDSVLLVTREDYFSCNTKNPIQSLTEGDSLFTFDRSGPFFFITGNADNCKKGQKLIVVVMAVRHKPQQQ

SEQ ID NO: 27 - protein, 15 aa, Artificial Sequence
  Synthetic peptide
KEIMVGGKTGAWKIP

(19) United States
(12) Patent Application Publication
(10) Pub. No.: US 2008/0124800 A1
(43) Pub. Date: May 29, 2008

(54) Arabinogalactan Protein Compositions and Methods for Fostering Somatic Embryogenic Competence

(75) Inventors: Simon Poon, Collingwood (AU); Robyn Louise Heath, Clifton Hill (AU); Adrienne E. Clarke, Parkville (AU)

Correspondence Address: GREENLEE WINNER AND SULLIVAN P C, 4875 PEARL EAST CIRCLE, SUITE 200, BOULDER, CO, 80301, US

(73) Assignee: Hexima Limited, Victoria (AU)

(21) Appl. No.: 10/594,418

(22) PCT Filed: Mar. 31, 2005
(86) PCT No.: PCT/IB05/01771

§ 371 Date: Dec. 28, 2006

Related U.S. Application Data
(60) Provisional application No. 60/558,609, filed on Mar. 31, 2004.

(51) Int. Cl. C12N 5/02; C07K 14/415; C12N 5/04

(52) U.S. Cl. 435/427; 435/430; 530/370

(57) ABSTRACT

Methods for fostering somatic embryogenic competence of a plant cell or tissue by contacting the plant cell or tissue with an arabinogalactan protein (AGP) composition effective for fostering somatic embryogenic competence in plant species and varieties that are recalcitrant to somatic embryogenesis and/or regeneration are provided. AGP compositions useful for fostering somatic embryogenic competence are provided, including total AGP and an AGP RP-HPLC hydrophobic fraction, both from embryogenic callus, from plant varieties including cotton lines Coker 315, Siokra 1-4, and Sicala 40, including at concentrations ranging from about 0.0008 mg/L to about 100 mg/L. Methods for regenerating plants, producing transformed plants, and impeding somatic embryogenic competence are also provided. Plant culture media and methods for making useful AGP compositions and media are also provided. Amino acid and nucleotide sequences for peptides and proteins eluting in hydrophobic AGP fractions are also provided.

### BACKGROUND OF THE INVENTION

Plant regeneration and transformation methods are known in the art (Razdan, M. K., Introduction to plant tissue culture, 2nd edition, Science Publishers, 2003; Plant cell culture protocols, edited by Robert D. Hall, Totowa, N.J., Human Press, 1999; Slater, Adrian et al., Plant biotechnology: the genetic manipulation of plants, Oxford; N.Y.: Oxford University Press, 2003; Genetic transformation of plants, edited by J. F. Jackson and H. F. Linskens, Publisher Berlin; New York: Springer, 2003; and Fehér, A. et al. (September 2003) Plant Cell, Tissue and Organ Culture 74(3):201-228).

Successful plant transformation is dependent on successful methods for plant regeneration. Plant species and varieties vary in their receptivity to plant regeneration techniques. While some species and varieties have been easy to regenerate, using many different protocols and tissues, others are recalcitrant to regeneration and have been very difficult (Benson E. E. (2000) In Vitro Cell. Dev. Biol.—Plant, 36:141-148). Plant species and varieties vary in how many and which tissues have pluripotential cells or cells that can become pluripotential cells, and under what conditions that developmental potential can be promoted. In most circumstances, regeneration potential is statistical, i.e., that a given tissue of selected variety of a selected species, in a selected environment, is likely to undergo a selected regenerative step at a certain frequency. When one species, variety, and/or tissue is less regenerable than another, the frequency at which it undergoes the regeneration step is lower. Species and varieties for which combinations of tissue types and environments undergo regeneration at a low frequency are said to be recalcitrant to regeneration. There is a need in the art for methods useful for regenerating agronomically useful plant species and varieties that are recalcitrant to regeneration.

One method for regenerating some plant species exploits the occurrence of somatic embryogenesis. Somatic embryogenesis differs from zygotic embryogenesis in that explants of somatic cells, that have not undergone meiosis, are induced to dedifferentiate to an embryogenic state and to form an embryo which can develop into a fertile plant. Dedifferentiation has been reported to require several rounds of cell divisions (Bai et al., (2000) Current topics in Developmental Biology 50:61-88). Unlike zygotic embryos, somatic embryos have the same genetic material as the somatic cells from which they arise. Often, explants are first induced to form callus, and then the callus is provided appropriate environmental conditions to promote somatic embryogenesis. Somatic embryogenesis is a stochastic process in which the variables affecting efficiency have not been completely defined. Increasing the efficiency of somatic embryogenesis includes increasing the likelihood, percentage, number, or rate of somatic embryos formed from a given number of explants.

Although cotton transformation methods are known in the art, cotton has traditionally been recalcitrant to regeneration (Wilkins et al. (2000) Crit. Rev Plant Sci, 19:511-550). Cotton is an agronomically important crop. The value of worldwide cotton production is over $20 billion annually, and the combined production, marketing, consumption, and trade of cotton-based products is over $100 billion annually in only the United States. Cotton is grown primarily for its lint, which provides high quality fiber for the textile industry. Cotton seed is also a valuable commodity, providing a source for oil, meal, and seed hulls. Cotton and cotton by-products provide raw materials that are used for foodstuffs, livestock feed, fertilizer, and paper. Several cotton species are grown, but about 90% of cotton grown worldwide is Gossypium hirsutum L., or Upland cotton, and about 8% is Gossypium barbadense, or Pima cotton. Other cotton species include Sea Island cotton and Egyptian cotton. There are over 7000 cotton accessions at the National Cotton Germplasm Collection (Germplasm Resources Information Network—GRIN, Cotton Collection, Curator: Percival, A. E. (713-260-9311), USDA-ARS, Texas &M University, P.O. Box DN—Cotton Genetics Research, College Station, Tex. 77841).

Cotton has historically been susceptible to a variety of pests. Cotton produces a sweet nectar that attracts a variety of destructive insect pests, including the boll weevil, bollworm, armyworm, and the red spider. In addition to insect pests, there is also a very destructive fungus, called the wilt, that attacks the root system of the cotton plant. There is a demand for transformed cotton, including cotton that is genetically engineered to be resistant to pests, disease, or herbicides, to have a higher yield, or to have an altered composition.

Although cotton regeneration protocols have been described, the only commercially successful protocols have required the use of Coker varieties, which have responded in tissue culture but are not agronomically important for many reasons including that they are susceptible to Fusarium wilt (Chlan et al. (1995) Plant Mol. Biol. Rep, 13(1):31-37; Firoozabady et al. (1987) Plant Molecular Biology 10:105-116; Peeters et al. (1994) Plant Cell Rep, 13:208-211; Shoemaker et al. (1986) Plant Cell Rpt, 3:178-181; Umbeck et al. (1987) Bio/Technology, 5:263-266; U.S. Pat. No. 4,672,035 (issued Jun. 9, 1987); WO 00/53,783 (published Sep. 14, 2000)). Consequently, it has been necessary to backcross all engineered traits in a successfully transformed Coker plant to elite varieties for many generations. Backcrossing transformation protocols require several years and many have ultimately failed due to the introgression of one or more poor agronomic traits along with the trait of interest. All currently available commercial transgenic cotton varieties are based on Coker transformation (Sakhanokho et al. (2001) Crop Sci., 41:1235-1240). More than 75% of cotton acreage in the U.S. is genetically modified cotton (Wilkins et al. (2001)). There has been a decline over the last decade in cotton yield and quality due to a decrease in genetic diversity of cotton planted (Bowman et al. (1996) Crop Sci, 36:577-581 and Meredith (2000) Proc World Cotton Res Conf 11, Athens, Greece, 97-101). There is a need in the art for increased cotton yield and increased genetic diversity in cotton.

Methods for regenerating Coker varieties have described the production of embryogenic callus from explants, the occurrence of somatic embryos, and subsequent germination and growth into mature cotton plants (Firoozabady and DeBoer (1993) In Vitro Cell Dev. Biol, 29 P:166-173; Firoozabady et al. (1987); Peeters et al. (1994); Hudspeth et al., (1996) Plant Mol. Biol. 1996 June; 31(3):701-5; Shoemaker et al. (1986); Umbeck et al. (1987); Davidonis and Hamilton (1983) Plant Science Letters, 32:89-93; Trolinder and Goodin (1987) Plant Cell Reports, 6:231-234; U.S. Pat. Nos. 5,159,135 (issued Oct. 27, 1992), 5,004,863 (issued Apr. 2, 1991) 5,244,802 (issued), 6,483,013 (issued Nov. 19, 2002), and 5,846,797 (issued Dec. 8, 1998); and WO 00/36911 (published 29 Jun. 2000)). The efficiency of somatic embryogenesis in cotton has been relatively low (Voo et al. (1991) In vitro Cell Dev Biol, 27 P:117-124; Zhang et al. (1993) Acta Agricultural Bioreali-occidentalis Sinica 24(4):24-48; Zhang and Zhao (1997) Cotton Biotechnology and Its Application, China Agricultural Press, Beijing).

Although methods for regenerating cotton species and varieties other than Coker have been described [Zhang et al. (2000) Plant Cell, Tissue and Organ Culture, 60:89-94; Sakhanokho et al. (2001); Zhang et al. (2001a) Bot. Bull. Acad. Sin, 42:9-16; Cousins Y L, Lyon B R, Llewellyn D J (1991) Aust. J. Plant Physiol. 18:481-494; U.S. Pat. Nos. 6,479,287 (issued Nov. 12, 2002), 5,859,321 (issued Jan. 12, 1999), 5,834,292 (issued Nov. 10, 1998), 5,874,662 (issued Feb. 23, 1999), 6,624,344 (issued Sep. 12, 2003), 6,573,437 (issued Jun. 3, 2003), 6,620,990 (issued Sep. 16, 2003), 5,244,802 (issued Sep. 14, 1993), 5,583,036 (issued Dec. 10, 1996), 5,695,999 (issued Dec. 9, 1997); EP 0317512 (published Aug. 5, 1992); WO 00/77230 (published Dec. 21, 2000); U.S. Patent Application No. 2003/0143744 (published Jul. 31, 2003); and WO 01/00785 (published Jan. 4, 2001)], they have either utilized Coker genetics (Mishra et al. (2003) Plant Cell, Tissue and Organ Culture, 73:21-35) or have not been shown to be useful for high efficiency regeneration of a broad range of elite cotton varieties. There is a need to obtain a sufficiently high regeneration efficiency, in part, because many plants regenerated from callus are not normal (Stelly et al. (1985) Agro Abstracts American Society of Agronomy p. 135).

There remains a need in the art for high efficiency methods for regenerating a broad range of elite cotton varieties. For example, none of the above-mentioned references have demonstrated somatic embryogenesis of Sicala 40.

Arabinogalactan proteins (AGPs) have been described as a family of structurally related, extensively glycosylated, hydroxyproline-rich glycoproteins (HRGPs) analogous to animal proteoglycans (Nothnagel E A, Bacic A, Clarke A E (Eds) (2000) Cell and developmental biology of arabinogalactan-proteins. Kluwer Academic/Plenum Publishers Corp, NY; Showalter (2001) Cell Mol Life Sci 58:1399-1417; and U.S. Pat. Nos. 6,350,594, 5,133,979, 5,296,245, 5,747,297, 6,271,001, 5,646,029, and 5,830,747). AGPs have been shown to be expressed throughout the plant kingdom and have been considered to have important roles in plant growth and development.

AGPs have been described as containing high proportions of carbohydrate and usually less than 10 percent by weight of protein [Clarke et al. (1978) Aust. J. Plant Physiol. 5:707-722; Fincher et al. (1983) Ann. Rev. Plant Physiol. 34:47-70], although AGPs having a protein content of about 59% have been reported [Fincher et al. (1983); Anderson et al. (1979) Phytochem. 18:609-610]. Reports have shown that the carbohydrate consisted of 30 to 150 unit polysaccharide chains, attached to multiple sites on the protein backbone, having a 1,3-β-D-galactopyranosyl backbone and side chains of (1,3-β- or 1,6-β-)D-galactopyranosyl (Galp) residues and often terminating in β-D-Galp and α-L-arabinofuranosyl (Araf) residues [Kreuger et al. (1993) Planta 189:243-248]. Other neutral sugars and uronic acids have also been detected, although at low levels. Monosaccharides which have also been demonstrated include L-rhamnopyranose, D-mannopyranose, D-xylopyranose, D-glucopyranose, D-glucuronic acid and its 4-0-methyl derivative and D-galacturonic acid and its 4-0-methyl derivative [Clarke et al. (1979) Phytochemistry 18: 521-540; Nothnagel (1997) Int Rev Cytol 174: 195-291; and Fincher et al. (1983)]. Short arabinose side chains have also been found on some AGPs.

AGPs have often been defined by their ability to react with the phenylazoglycoside dye called Yariv reagent (Yariv et al. (1962) Biochem J 85:383-388 and Yariv et al. (1967) Biochem J 105:1c-2c). Many protein backbones of AGPs have been cloned, their protein sequences and carbohydrate content analyzed (Showalter (2001) and U.S. Pat. Nos. 6,350,594, 5,133,979, 5,296,245, 5,747,297, 6,271,001, 5,646,029, and 5,830,747).

Historically AGPs have been divided into two groups, classical and non-classical. Classical AGPs have been defined by protein sequence characteristics. They have been described to contain hydroxyproline (Hyp), Ala, Ser, Thr, and Gly as major amino acid constituents. Non-classical AGPs have been reported to be different in a variety of ways, such as having a low Hyp content, a high Cys content, or a high Asn content, for example. Reports have shown that classical AGPs typically have a hydrophobic C-terminal tail and can be glycosylphosphatidylinositol (GPI)-anchored to cell membrane proteins. AGPs have been categorized as one subclass of a larger class of proteins called Pro-/Hyp-rich glycoproteins (P/HRGPs), that also has been described to include Pro-rich proteins (PRPs) and extensins. Recently a new nomenclature for P/HRGPs was proposed (Schultz et al. (2002) Plant Physiology 129:1448-1463). If an AGP protein backbone contains several different regions, it would be called chimeric if one region is unrelated to P/HRGP motifs, and it would be called hybrid if one motif is of a different P/HRGP type. Using this system, most non-classical AGPs would be labeled as chimeric AGPs.

AGPs have been shown to be expressed in leaves, stems, roots, floral structures, and seeds (Fincher et al. (1983) and Nothnagel (1997)), with individual AGP family members exhibiting organ and tissue specific patterns of developmentally and environmentally regulated expression. AGPs have been localized to plasma membranes, cell walls (Minorsky, P. V., (February 2002) Plant Physiology 128:345-353), intercellular spaces, and secreted to the outside environment. AGPs have been suggested to be markers of cellular identity and fate. They have appeared to be associated with growth of leaf primordia, xylem development, secondary cell wall thickening, wound healing, programmed cell death, and embryogenesis (Majewska-Sawka, A. et al. (2000) Plant Physiol. 122:3-9.

In a few species, AGPs have been suggested to be involved in embryogenesis. Steele-King et al. (2000) Cell and Developmental Biology of Arabinogalactan-Proteins Chapter 9, ed. Nothnagel et al. Kluwer Academic/Plenum Publishers, 95-107 described the association of AGPs with producing the plant body, cell proliferation, and cell differentiation. Steele-King et al. described that addition of 5 μM Yariv reagent to proembryonic carrot masses resulted in a three- to fourfold increase in fresh weight of material, but that addition of 30 μM Yariv reagent did not. Chapman et al. (2000) Plant 211:305-314 described using Yariv reagent to block somatic embryogenesis in two Cichorium species. Egertsdotter and Arnold (1998) J of Exp Bot 49(319):155-162 reported using extracts of mature spruce seeds to stimulate or inhibit embryo development in Picea abies (Norway spruce). In spruce, the mature seed extract was reported to be capable only of stimulating embryos that had reached a certain size. Mature spruce seed extracts were described to contain chitinase-like proteins. Chitinases are enzymes that hydrolyze 13 (1-4) linkages between adjacent N-acetyl-D-glucosamine (GlcNAc) residues. A chitinase 4-related chitinase was described to have a stimulating effect on early embryo development, but to not affect later stages of embryo development. Extracts of immature seeds did not have any positive influence on embryo development.

Kreuger et al. (1993) reported that the addition of an AGP preparation from a Daucus carota L. (carrot) non-embryogenic cell line, initiated the development of an explant culture to become non-embryogenic. It was also reported that carrot cells developed into embryogenic cell lines regardless of the addition of carrot seed AGPs. Concentrations of 10 to 100 nM were described. Kreuger et al. (1995) Planta 197:135-141 described using antibodies to isolate specific carrot and tomato AGP fractions. One AGP fraction (ZUM 15) was described to induce vacuolation of embryogenic cells that then failed to produce embryos. Another fraction (ZUM 18) was described to increase the percentage of embryogenic cells. Fractions containing both ZUM 15 and ZUM 18 epitopes showed no embryogenesis promoting activity. The optimum concentration of ZUM 18 AGPs was said to be 0.2 mg/L. It was stated that any response to addition of AGPs was the result of adding a mixture of AGPs. This result reflects the heterogeneity of AGPs that makes it difficult to test individual components.

Toonen et al. (1997) Planta 203:188-195 reported that the addition of ZUM 18 AGP fraction to different size-fractionated cell populations from embryogenic carrot suspension cultures did not have a significant effect on the frequency and the morphology of the somatic embryos produced. An AGP fraction containing the JIM 8 epitope appeared to have an inhibitory effect. Addition of carrot seed AGPs to non-embryogenic cultures did not promote embryogenic competence, except after enrichment for cell clusters and removal of single vacuolated cells.

Zhang B H, et al. (2001b) Isr. J. Plant Sci. 49:193-196 described somatic embryogenesis and plant regeneration from cotton explants. Contacting explants with 2,4-D (auxin) prohibited the formation of embryogenic callus.

van Hengel et al. (2001) Plant Physiology 125:1880-1890 and van Hengel (1998) Chitinases and AGPs in Somatic Embryogenesis, Ph.D. thesis, Wageningen Agricultural University Wageningen, The Netherlands reported that carrot protoplasts, compared to carrot cells having cell walls, showed a reduced capacity for somatic embryogenesis that could be partially restored by adding endochitinases (EP3), that could be fully restored or increased by adding AGPs from culture medium or immature seeds. AGPs pretreated with chitinases were reported to be even more active in restoring capacity for somatic embryogenesis. AGPs were stated to require an intact carbohydrate constituent for activity. AGPs were also associated with re-initiation of cell division in a subpopulation of non-dividing protoplasts. van Hengel et al. (2002) Physiologia Plantarum 114:637-644 reported that the capacity to increase the frequency of somatic embryogenesis was observed to occur only with AGPs that were isolated from seeds in which the endosperm had been cellularized.

Kreuger M. et al. (2000) Cell and Developmental Biology of Arabinogalactan-Proteins Chapter 10, ed. Nothnagel et al. Kluwer Academic/Plenum Publishers, 109-119 reviewed effects of AGPs and chitinases on somatic embryogenesis. Kreuger et al. (2000) reported that although AGPs were thought to play a role in somatic embryogenesis, it was unclear whether the reported effects were due to a single AGP, mixtures of AGPs, the entire intact AGP molecule, or AGP fragments. This reference also stated that an unanswered question was whether the changes in AGP compositions were cause or effect. The reference also reported that to obtain an embryogenic cell line from an explant, it was essential to provide a correct auxin supply, that the timing of different events was crucial, and that an imbalance caused a different morphogenic path, e.g., root formation. The reference stated that the carbohydrate part of AGPs was responsible for increasing the frequency of somatic embryogenesis, and that AGPs were only capable of promoting embryogenesis when present during a critical time. This reference proposed that the biological effect of activated GlcNAc-containing AGPs was to maintain the embryo identity of both the somatic and zygotic embryo.

PCT publication WO 01/41557, published Jun. 14, 2001, described methods for enhancing embryogenesis from microspores using AGP, auxin, and ovary co-culture, but no organism or tissue source of AGP was given.

None of the above-mentioned references described a method using AGP derived from embryogenic callus.

European patent application publication number 0 455 597 A1, published on Nov. 6, 1991, alleged that adding AGPs to a culture medium stimulated growth division or somatic embryogenesis of plant cells. No experimental evidence or data was provided, and no active AGP components were identified.

None of the above-mentioned references has described a method using cotton as a source of AGP or for affecting cotton somatic embryogenesis. None of the above-mentioned references described AGP methods or compositions useful for affecting somatic embryogenesis in a species that is recalcitrant to regeneration. None of the cited references have physically or chemically characterized AGP fractions that enhance or stimulate somatic embryogenesis. None of the above-mentioned references described hydropathic fractionation of AGPs or activity of AGP hydropathic fractions. None of the above-mentioned references described using AGP without arabinose and/or other glycosylation.

All references cited are incorporated herein by reference in their entirety to the extent that they are not inconsistent with the disclosure herein.

### SUMMARY OF THE INVENTION

This invention provides methods for fostering somatic embryogenic competence and arabinogalactan compositions useful for performing these methods.

This invention provides methods for fostering somatic embryogenic competence of a plant cell or tissue or progeny thereof comprising contacting the plant cell with an arabinogalactan protein (AGP) composition effective for fostering somatic embryogenic competence, increasing the likelihood that a cell will undergo somatic embryogenesis, improving the efficiency of somatic embryogenesis, increasing the number or percentage of a plurality of plant cells or tissues producing embryogenic explants over time, and/or decreasing the time until a plant cell or tissue undergoes somatic embryogenesis including the formation of proembryonic masses and/or embryogenic callus, relative to a selected standard.

This invention provides methods for fostering somatic embryogenic competence using a pro-embryogenic AGP composition from the same species, the same variety, a different species, or from a more embryogenic variety of the same species.

This invention provides methods for fostering somatic embryogenic competence wherein the plant cell or tissue and/or the source of the pro-embryogenic AGP composition is a dicot, a monocot, an agronomically useful plant, a fiber-producing plant, of the Order Malvales, or of a species and/or variety that is recalcitrant to regeneration. This invention provides methods for fostering somatic embryogenic competence wherein the plant cell or tissue and/or the source of the pro-embryogenic AGP composition is a cotton cell or tissue. This invention provides methods for fostering somatic embryogenic competence in a broad range of elite cotton varieties.

This invention provides methods for fostering somatic embryogenic competence including wherein the plant cell or tissue is an explant from a plant or a callus cell or tissue derived from an explant. In an embodiment of this invention, the AGP composition is derived from embryogenic callus, proembryonic masses, and/or embryos.

In embodiments of this invention, the AGP composition useful for fostering somatic embryogenic competence comprises a concentration of between about 0.01 mg/L and about 100 mg/L, between about 0.05 mg/L and about 50 mg/L, between about 0.08 mg/L and about 30 mg/L, between about 0.1 mg/L and about 20 mg/L, between about 0.5 mg/L and about 10 mg/L, between about 1 mg/L and about 4 mg/L AG, and/or between about 1 mg/L and about 2 mg/L AGP or total AGP.

In embodiments of this invention, the AGP composition comprises purified AGP, is purified by Yariv reagent extraction, comprises total AGP, is further purified or fractionated by a hydropathic separation methodology, is further purified by reverse phase high performance liquid chromatography (RP-HPLC), is not purified using an antibody, comprises a hydrophobic AGP fraction, and/or comprises hydrophobic peak #1.

In an embodiment of this invention, the AGP composition comprises the hydrophobic AGP fraction at a concentration of between about 0.0015 mg/L and about 15 mg/L AGP. In an embodiment of this invention, the AGP composition comprises hydrophobic peak #1 at a concentration of between about 0.0008 mg/L and about 8 mg/L AGP.

AGP compositions useful in the practice of this invention include unextracted and unpurified cell lysate, Yariv reagent extracted AGP, total AGP, purified AGP, fractionated AGP, deglycosylated AGP, dearabinosylated AGP, deglycosylated and dearabinosylated AGP, AGP with and without post-translational modification, hydrophobic AGP fractions, hydrophobic AGP peaks #1, #2, and #3 protease treated AGP, AGP peptide fragments, engineered AGP that is arabinosylated and/or glycosylated, AGP that is differently arabinosylated and/or glycosylated, engineered AGP that is not arabinosylated and/or glycosylated, and chemically synthesized AGP.

In an embodiment of this invention, the percentage of explants producing embryogenic callus is increased by at least about 20%, at least about 50%, at least about 75%, or at least about 100%. In an embodiment of this invention, contacting the cell or tissue with an AGP composition effective for fostering somatic embryogenic competence decreases the time until the plant cell or tissue undergoes somatic embryogenesis by about two weeks, by at least 25%, or by at least 50% relative to not contacting with an AGP composition.

In an embodiment of this invention, the plant cell or tissue is in culture in contact with a culture medium having 0.5 mg/L kinetin and 1 mg/L indole-3-butyric acid, or was previously in contact with such a culture medium. In an embodiment of this invention, the culture medium contains the AGP composition.

This invention provides somatic embryos produced by the method for fostering somatic embryogenic competence. This invention provides somatic embryogenic callus produced by the methods of this invention.

This invention provides a method for regenerating a plant comprising harvesting a plant cell or tissue from a first plant; contacting the plant cell or tissue with an AGP composition effective for fostering somatic embryogenic competence; and regenerating a second plant from the plant cell or tissue. This invention provides plants and progeny produced by the above-described method. This invention provides seeds produced by the above-described plants and progeny.

This invention provides a method for transforming a plant comprising: harvesting a plant cell or tissue from a plant; transforming the plant cell or tissue; contacting the transformed plant cell or tissue with an AGP composition effective for fostering somatic embryogenic competence; and regenerating a transformed plant from said plant cell or tissue. This invention provides transformed plants and progeny produced by the above-described method. This invention provides seeds produced by the above-described transformed plants and progeny.

This invention provides a method for making an AGP composition useful for fostering somatic embryogenic competence comprising: providing embryogenic callus; and harvesting AGP from said embryogenic callus. This invention provides a method for making an AGP composition useful for fostering somatic embryogenic competence comprising: expressing a protein or peptide comprising an amino acid sequence selected from the group consisting of SEQ ID NOS:1-7, 15, 17, and portions thereof; and harvesting the protein or peptide.

This invention provides a method for making a plant cell culture medium effective for fostering somatic embryogenic competence comprising: providing a plant cell culture medium; and adding an AGP composition effective for fostering somatic embryogenic competence.

This invention provides a method for impeding somatic embryogenic competence in a plant cell or tissue comprising contacting the plant cell or tissue with an AGP composition effective for impeding somatic embryogenic competence, compared to not contacting the plant cell or tissue with the AGP. In embodiments of this invention, the AGP composition comprises total AGP from non-embryogenic callus, the hydrophilic AGP, hydrophilic peak #1, AGP derived from a variety that is less embryogenic than the plant cell or tissue, and mixtures thereof.

This invention provides a method for maintaining a plant cell or tissue in culture comprising contacting the plant cell or tissue with an AGP composition effective for plant cell or tissue maintenance.

This invention provides a method for fostering callus formation in a plant cell or tissue comprising contacting said plant cell with an AGP composition effective for fostering callus formation.

This invention provides a method for culturing a plant cell comprising contacting said plant cell with a culture medium comprising 0.5 mg/L kinetin and 1 mg/L indole-3-butyric acid.

This invention provides a method for fostering somatic embryogenic competence in a plant cell or tissue comprising contacting said plant cell with a composition comprising 0.5 mg/L kinetin and 1 mg/L indole-3-butyric acid.

This invention provides a purified AGP composition effective for fostering somatic embryogenic competence of a plant cell or tissue.

In an embodiment of this invention, the pro-embryogenic AGP composition comprises a protein or peptide having a sequence of SEQ ID NO: 15 or SEQ ID NO:17, capable of being encoded by SEQ ID NOS: 14 or 16, a portion of at least fifteen amino acids of SEQ ID NO: 15 or 17, or having at least 80% sequence similarity to SEQ ID NOS: 15 or 17, or a protein having at least 80% sequence similarity to SEQ ID NOS: 25 or 26 or a tryptic digest thereof.

In an embodiment of this invention, the pro-embryogenic AGP composition comprises a peptide having a sequence of SEQ ID NOS: 1-7.

### BRIEF DESCRIPTION OF THE DRAWINGS

FIG. 1 is a chart showing quantitation, by absorbance at 215 nm, of embryogenic AGPs eluting off of a RP-HPLC column over time in minutes, as described in Example 3. Non-embryogenic AGPs are designated by a dashed line and pro-embryogenic AGPs by a solid line.

FIG. 2 is a graph showing the percentage of embryogenic explants after four, six, and eight weeks of contact with embryogenic AGP, for eight trials, as described in Example 4. The control, no contact with embryogenic AGP, is striped, and contact with embryogenic AGP is solid.

FIG. 3 is a graph showing the percentage of embryogenic explants after four, six, and eight weeks of contact with non-embryogenic AGP, for four trials, as described in Example 6. The control, no contact with non-embryogenic AGP, is striped, and contact with non-embryogenic AGP is solid.

FIG. 4 is a graph showing the percentage of embryogenic explants after four, six, and eight weeks of contact with AGP, for five trials, as described in Example 7. The control, no contact with AGP, is striped, and contact with gum Arabic AGP is solid.

FIGS. 5A and 5B are graphs showing the percentage of embryogenic explants after four, six, and eight weeks of contact with a range of concentration % total AGP from embryogenic callus for two trials, as described in Example 8. Data from trial #1 are graphed in FIG. 5A, data from trial #2 are graphed in FIG. 5B. Results of control (diagonally striped), 1 mg/L (grid), 2 mg/L (solid), and 4 mg/L (horizontally striped) embryogenic callus AGP are shown.

FIG. 6 is a chart showing quantitation, by absorbance at 215 nm, of embryogenic callus AGPs eluting from a RP-HPLC column over time in minutes, as described in Example 9. The vertical line at 15 minutes, about 20% acetonitrile, denotes a separation between the hydrophilic (left-pointing arrow) and hydrophobic (right-pointing arrow) fractions.

FIG. 7 is a graph showing the percentage of embryogenic explants after four, six, and eight weeks of contact with fractionated AGP, for three trials, as described in Example 10. The control, no contact with AGP, is striped, 0.85 mg/L of the hydrophilic fraction has no fill, and 0.15 mg/L of the hydrophobic fraction is solid.

FIG. 8 is a chart showing quantitation, by absorbance at 215 nm, of embryogenic callus AGPs eluting off of a RP-HPLC column over time in minutes, as described in Example 11. Four peaks are labeled. Time points used to begin and end collection of each peak are shown.

FIGS. 9A and 9B are graphs showing the percentage of embryogenic explants after four, six, and eight weeks of contact with an embryogenic AGP, for two trials, as described in Example 12. Trial #1 data is graphed in FIG. 9A, trial #2 data is graphed in FIG. 9B. The control (no contact with AGP) is diagonally striped, Fraction 1 has no fill, Fraction 2 has a grid, Fraction 3 is solid, and Fraction 4 is horizontally striped.

FIGS. 10A and 10B are graphs showing the percentage of embryogenic explants after four, six, and eight weeks of contact with dearabinosylated or deglycosylated embryogenic callus total AGP, for two trials, trial #1 shown in FIG. 10A and trial #2 FIG. 10B as described in Example 14. The control (no contact with AGP) is diagonally striped, dearabinosylated AGP (TFA treated) is solid, and deglycosylated AGP HF treated) has no fill.

FIG. 11 is a graph showing the percentage of Siokra 1-4 embryogenic explants after four, six, and eight weeks of contact with Coker 315 total embryogenic callus AGP, as described in Example 16. The control (no contact with AGP) is diagonally striped, and AGP is solid.

FIG. 12 shows an illustration of the protein domain structure of the AGP backbone having sequences of SEQ ID NOS:8 or 9, as described in Example 25. The AGP is divided into four domains: signal sequence (1), phytocyanin-like (2), pro-rich (3), and hydrophobic C-terminal (4).

FIG. 13 is a chart showing quantitation, by absorbance at 215 nm, of pro-embryogenic AGPs eluting off of a RP-HPLC column over time in minutes, as described in Example 27. Siokra 1-4 AGPs are designated by a dashed line and Coker 315 AGPs by a solid line.

FIG. 14 shows an amino acid sequence alignment of SEQ ID NOS: 15 and 17, as described in Example 25.

### DETAILED DESCRIPTION OF THE INVENTION

As used herein, “somatic embryogenic competence of a plant cell or tissue” refers to the likelihood a plant cell or tissue, or progeny thereof will develop into or give rise to somatic embryos, proembryonic masses, and/or embryogenic callus, such structures being identifiable by those skilled in the art. A “plant tissue” as used herein includes any collection of plant cells, including differentiated, undifferentiated, dedifferentiated cells or mixture thereof, whether living in vivo as part of a whole plant or living in vitro culture as an explant, undifferentiated callus, pro-embryogenic callus or somatic embryo, all as understood in the art. As used herein, “fostering somatic embryogenic competence” refers to promoting the efficiency of somatic embryo production by a plant cell or tissue, including increasing the likelihood that the cell or tissue will develop to form a somatic embryo, increasing the number or percentage of a plurality of plant cells or tissues producing somatic embryos over time, and/or decreasing the time until a plant cell or tissue undergoes somatic embryogenesis wherein somatic embryogenesis includes the formation of proembryonic masses and/or embryogenic callus; relative to a selected standard. Typically, the selected standard will be the same procedure except for attempting to obtain somatic embryogenesis with addition of an AGP. As used herein, “undergo somatic embryogenesis” refers to a plant cell or tissue, or progeny thereof, including a callus cell or tissue, developing into one or more art recognizable somatic embryos, or proembryonic masses, or embryogenic callus, during incubation in appropriate culture conditions. Callus, as is known in the art, is a plant tissue containing less differentiated or de-differentiated plant cells, such as can result from a wound. Callus tissue and cells can have the potential to follow many developmental fates, including programmed cell death, depending on many factors, including the environment in which they are cultured. Callus types include embryogenic callus and non-embryogenic callus.

As used herein, “impeding somatic embryogenic competence” refers to reducing the efficiency of somatic embryogenesis, decreasing the likelihood that a cell will undergo somatic embryogenesis, decreasing the number or percentage of a plurality of plant cells or tissues producing somatic embryos over time, and/or increasing the time until a plant cell or tissue undergoes somatic embryogenesis including the formation of proembryonic masses and/or embryogenic callus; relative to a selected standard.

As used herein, “embryogenic AGP is AGP obtained from embryogenic callus. As used herein, “pro-embryogenic AGP” refers to an AGP composition effective for fostering somatic embryogenic competence. As demonstrated herein, embryogenic AGP has the activity of fostering somatic embryogenic competence. As used herein, “non-embryogenic AGP” refers to an AGP composition that is not effective for fostering somatic embryogenic competence or that impedes somatic embryogenic competence.

As used herein, and in the art, “embryogenic callus” refers to plant tissue competent to form somatic embryos, including plant tissue from which somatic embryos can develop or are developing. Embryogenic callus includes callus containing proembryonic masses, callus in which there are no detectable embryos, and callus having detectable embryos. “Proembryonic masses” is used as in the art, includes cells that are on a developmental pathway into embryos. As used herein, “non-embryogenic callus” refers to plant tissue having no somatic embryos and in which no proembryonic masses are detectable by those of skill in the art. Non-embryogenic callus is not detectably competent to form somatic embryos. Non-embryogenic callus includes callus grown under conditions known to produce no somatic embryos or proembryonic masses and callus which has not as yet produced somatic embryos or proembryonic masses or does not as yet have other physical characteristics of embryogenic callus, even though grown in conditions known to produce somatic embryogenesis or pro-embryonic masses on occasion.

As used herein, “fostering callus formation” refers to increasing the number or percentage of a plurality of plant cells or tissues (explants) producing callus over time or decreasing the time until a plant cell or tissue undergoes callus formation, relative to a standard, wherein the callus can include non-embryogenic callus, embryogenic callus, and mixtures thereof.

As used herein, and in the art, “maintaining a plant cell or tissue in culture” refers to maintaining a living status of a plant cell or tissue while in tissue culture and to maintaining a selected developmental potential of the cell or tissue. “Culturing a plant cell” is used as understood in the art and includes maintaining a plant cell, providing nutrients (e.g. light, sugars, hormones, and/or vitamins), providing conditions allowing for growth and/or development of that cell, including by in vitro culturing, on soil, on solid media, and in liquid media. “In culture” is used as understood in the art and includes in vitro culture, culture on a solid medium, and suspension culture.

An explant is scored as embryogenic when embryogenic callus can be detected on it or in it by one of skill in the art. The total number of explants having at least one section of embryogenic callus scored at a given time point divided by the total number of explants scored is the percentage of explants that are embryogenic.

“Plant cell culture medium” is used as in the art and includes dehydrated media, concentrated media, liquid media, and solid media.

As used herein, “more embryogenic variety” refers to a plant variety that, under identical environmental conditions, produces more embryogenic callus or somatic embryos or produces embryogenic callus or somatic embryos more quickly than another variety of the same species.

As used herein, “cell types useful for producing callus” include all plant cell types capable of producing callus using methods known in the art, methods of this invention, and methods as yet to be discovered. Cell types useful for producing callus include cell types in: roots, shoots, stems, hypocotyls, transition regions, leaves, cotyledons, stomata, petioles, anthers, microspores, flowers, primordia, and apices.

As used herein and in the art, arabinogalactan protein (AGP) refers to a class of plant products composed of a protein that is post-translationally modified by glycosylation and/or arabinosylation. As found in nature, AGPs can be precipitated by Yariv reagent. The terms “Yariv precipitable material” and “AGP” are often considered synonymous. A wide variety of AGPs exist in nature, the exact structures and relative abundance of AGPs differing among plant species and among tissues of the same plant at different stages of development. Amino acid sequence analysis of the protein compound of certain purified AGPs has revealed structural differences among AGPs and has allowed for recognition of different sequence motifs that have similarities to other, non-AGP proteins. Of interest herein are phytocyanin-like (PL) domains of embryogenic AGPs described herein. Activity for fostering embryogenesis in cotton has now been found to be a property of the embryogenic AGP compositions described herein, the de-glycosylated/de-arabinosylated protein components thereof and of PL protein domains within the protein components. In view of the common activity of fostering embryogenic competence associated with naturally-occurring embryogenic AGP as well as its de-glycosylated/de-arabinosylated protein component and of at least one protein domain within the protein component, all such components are included within the term “pro-embryogenic AGP,” regardless of how they are made.

As used herein, an AGP composition “effective for fostering somatic embryogenic competence,” or alternatively, “pro-embryogenic AGP” refers to an AGP composition having an activity of promoting, or increasing the number or percentage of, a plurality of plant cells or tissues forming somatic embryos over time or decreasing the time until a plant cell or tissue undergoes somatic embryogenesis including the formation of proembryonic masses and/or embryogenic callus, relative to a standard treatment, e.g. not using the AGP composition, when the AGP composition is in contact with the cell(s) or tissue(s). “Contact” is used as in the art and includes fluid contact. “Regenerating a plant” is used as in the art and includes growing a fertile organism. An AGP composition extracted from embryogenic callus is sometimes denoted herein as “embryogenic AGP”.

As used herein, “total AGP” refers to a composition having all the types of AGP from a sample, i.e., from which no Yariv reagent binding AGP fraction has been previously removed.

As used herein, “hydrophilic AGP fraction” refers to a hydropathic fraction of an AGP composition which is relatively more hydrophilic than other fractions obtainable by a process that separates AGP's by their hydropathic character. An example of a hydrophilic AGP fraction includes a cotton AGP RP-HPLC fraction from callus that elutes, from a Brownlee Aquapore OD-300 7 μm reverse-phase HPLC column (2.1×100 mm) (Perkin Elmer, Wellesley, Mass., USA) that has been equilibrated in 0.1% v/v trifluoroacetic acid (TFA), using a linear gradient from 0% acetonitrile and 0.1% v/v TFA to 80% v/v acetonitrile, 0.089% v/v TFA over 60 min at a flow rate of 0.5 mL/min., or from at using a semi-preparative Zorbax 300 SB-C8 9.4 mm×25 cm column and a flow rate of 3 mL/min., between 0% and 20% acetonitrile and including a cotton AGP RP-HPLC fraction from embryogenic callus that consists essentially of a hydrophilic peak that elutes between 4-12% acetonitrile, and that comprises about 85% of total AGP quantity.

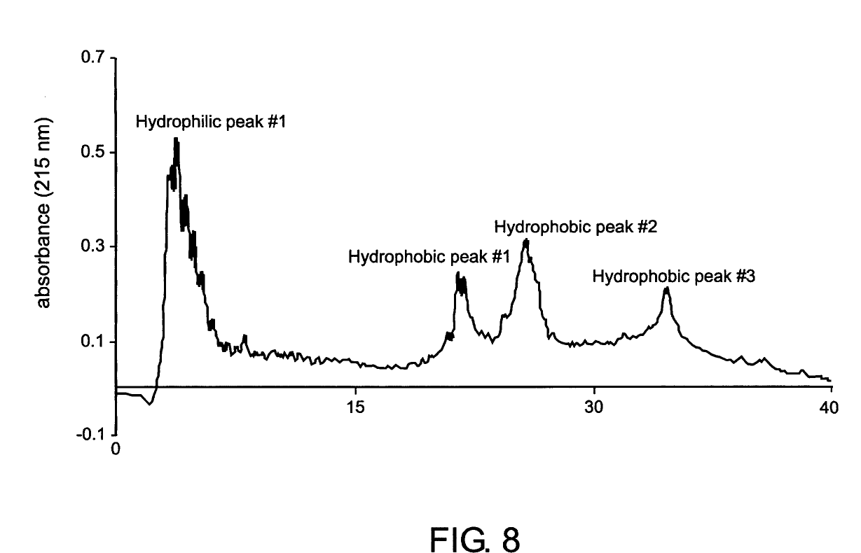

As used herein, and shown in FIG. 8, “hydrophobic AGP fraction” refers to a hydropathic fraction an AGP composition which is relatively more hydrophobic than other fractions obtainable by a process that separates AGPs by their hydropathic character. An example of a hydrophobic AGP fraction includes a cotton AGP RP-HPLC fraction from embryogenic callus that elutes between about 20% and 80% acetonitrile, that comprises about 15%-25% of total AGP quantity, and that includes a cotton AGP RP-HPLC fraction from embryogenic callus that consists essentially of hydrophobic peaks that elute between about 27-32% acetonitrile, about 32-37% acetonitrile, and about 44-49% acetonitrile.

As used herein, and shown in FIG. 8, “hydrophobic peak #1” (also termed Fraction 2 herein) refers to the AGP peak eluting between about 27-32% acetonitrile from an RP-HPLC column, from application of a cotton embryogenic callus total AGP composition. Equivalents of hydrophobic peak #1 (Fraction 2) include peaks eluting from an RP-HPLC column from application of an AGP containing composition from any tissue from any plant species wherein an AGP within the peak is capable of fostering somatic embryogenic competence, relative to total AGP from the same tissue of the same species.

As used herein, and shown in FIG. 8, “hydrophobic peak #2” (also termed Fraction 3 herein) refers to the AGP peak eluting between about 32-37% acetonitrile from an RP-HPLC column, from application of a cotton embryogenic callus total AGP composition. Equivalents of hydrophobic peak #2 (Fraction 3) include peaks eluting from an RP-HPLC column from application of an AGP containing composition from any tissue from any plant species wherein an AGP within the peak is capable of fostering somatic embryogenic competence, relative to total AGP from the same tissue of the same species.

As used herein, and shown in FIG. 8, “hydrophobic peak #3” (also termed Fraction 4 herein) refers to the AGP peak eluting between about 44-49% acetonitrile from an RP-HPLC column, from application of a cotton embryogenic callus total AGP composition. Equivalents of hydrophobic peak #3 (Fraction 4) include peaks eluting from an RP-HPLC column from application of an AGP containing composition from any tissue from any plant species wherein an AGP within the peak has comparable activity of fostering somatic embryogenic competence, as has been exemplified herein.

As used herein, and shown in FIG. 8, “hydrophilic peak #1” (also termed Fraction 1 herein) refers to the AGP peak eluting between about 4-12% acetonitrile from an RP-HPLC column, from application of a cotton embryogenic callus total AGP composition. Equivalents of hydrophilic peak #1 (Fraction 1) include peaks eluting from an RP-HPLC column from application of an AGP containing composition from any tissue from any plant species wherein an AGP within the peak does not foster somatic embryogenic competence and does impede somatic embryogenic competence, relative to total AGP from the same tissue of the same species.

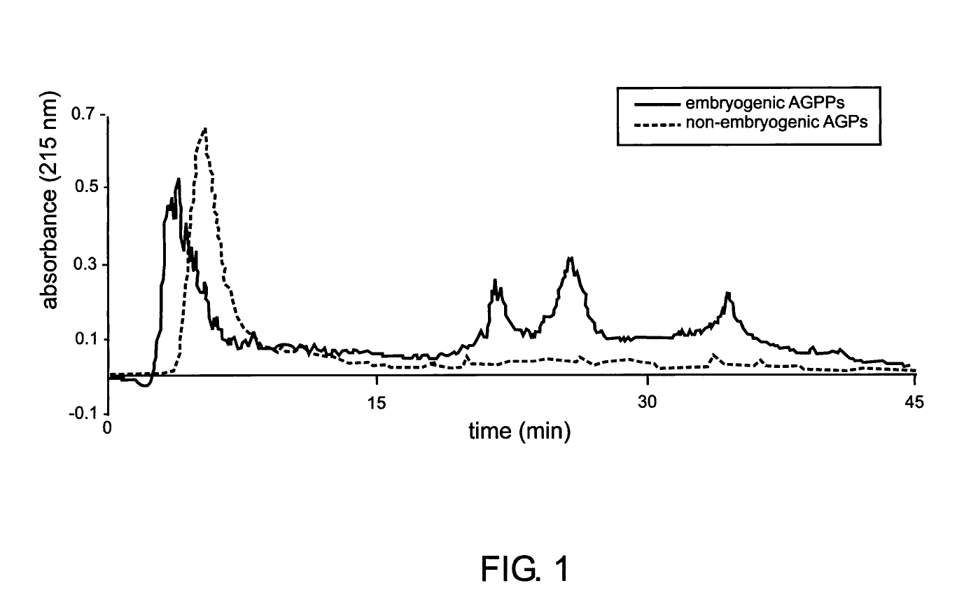

As used herein, and shown in FIG. 1, “non-embryogenic hydrophilic peak” refers to the AGP peak eluting between about 3-11% acetonitrile from an RP-HPLC column, from application of a cotton non-embryogenic callus total AGP composition. The non-embryogenic RP-HPLC profile comprises the peak and a tail. Equivalents of a non-embryogenic hydrophilic peak include peaks eluting from an RP-HPLC column from application of an AGP containing composition from any tissue from any plant species wherein an AGP within the peak does not foster somatic embryogenic competence and does impede somatic embryogenic competence, relative to total AGP from the same tissue of the same species.

“Coker cotton varieties” is used as in the art and is intended to include Coker 201, Coker 310, Coker 315, Coker 320, Coker 130, Coker 139, Coker 304, Coker 312, transgenic Coker, Coker varieties available at the National Cotton Germplasm Collection (Germplasm Resources Information Network), and varieties having at least about 50% Coker genetics.

“Acala cotton varieties” is used as in the art and is intended to include Acala MAXXA, Acala Riata, Acala Sierra, transgenic Acala, DP 6207 Acala, PHY 72 Acala, PHY 78 Acala, and varieties having at least about 50% Acala genetics, defined as being a first generation cross of a standard Acala variety such as one of those named. The progeny of further outcrosses are excluded from the definition of “Acala cotton varieties”.

“Agronomically useful plants” is used as in the art and is intended to include crops grown for fiber, grain, silage, fruit, vegetables, herbs, flowers, oil, sugar, including cotton, wheat, corn, soybean, cereals, beans, pulses, ornamentals, and tobacco as well as crops grown for timber, pasture, food additives, fragrances, medicines and pharmaceuticals, including citrus, poppies, grapevines, berries, apples, pears, sandalwood, echinaceae, pine, rice, barley and all plants that can be transformed.

The phrase, “fiber-producing plants” is used as in the art and is intended to include cotton, kenaf, milkweed, flax, hemp, nettle, hop, and milkweed.

“Elite cotton lines” is used as in the art and is intended to include Coker 315, Sicala 40, Siokra 1-4, Sicot 189, Emerald, Sicala 43, Sicala 45, Sicala V-2, Sicot 53, Sicot 70, Sicot 71, Sicot 80, Siokra S-102, Siokra V-16, Siokra V-17, Siokra V-18, Pearl, Sapphire, Topaz, Opal, Diamond, transgenic cotton varieties, Sicot 11B, Sicot 12B, Sicot 13B, Sicot 14B, PSC 355, 1517-77, 1517-95, 1517-99, Acala MAXXA, Acala Riata, Acala Sierra, AG 3601, Atlas, BXN 47, BXN 49B, DP 388, DP 422, DP 436, DP 449, DP 451, DP 458, DP 468, DP 5415, DP 555, DP 5690, DP 6207 Acala, DP 655, DP 655, FM 832, FM 958, FM 966, FM 989, FM 989, FM 991, HS 44, NuCOTN 33, NuCOTN 35, Paymaster HS 26, PHY 72 Acala, PHY 78 Acala, PM 1199, PM 1218, PM 1560, PM 2145, PM 2156, PM 2167, PM 2200, PM 2266, PM 2280, PM 2326, PM 2379, SG 215, ST 2454, ST 457, ST 4691, ST 4793, ST 4892, ST 4892, ST 5303, ST 5599, SG 105, SG 125, SG 501, SG 521, Xpress, and cotton varieties sold commercially.

Gum Arabic is a gummy exudation originating from the Acacia tree. Gum Arabic contains AGPs.

This invention provides a method for fostering somatic embryogenic competence of a plant cell or tissue or progeny thereof comprising contacting the plant cell with an arabinogalactan protein (AGP) composition effective for fostering somatic embryogenic competence. In an embodiment of this invention, fostering somatic embryogenic competence includes improving the efficiency of somatic embryogenesis, increasing the likelihood that a cell will form a somatic embryo, increasing the number or percentage of somatic embryogenic callus formed by a plurality of plant cells or tissues over time, and/or decreasing the time until a plant cell or tissue undergoes somatic embryogenesis including the formation of proembryonic masses and/or embryogenic callus, relative to a selected standard.

In an embodiment of this invention, a comparison standard is obtained by growing equivalent plant cells or tissue under the same conditions used for fostering somatic embryogenic competence, except for the absence of an AGP composition effective for fostering somatic embryogenic competence. In an embodiment of this invention, contacting a plant cell with an embryogenic AGP fosters somatic embryogenic competence compared to not contacting said plant cell with a pro-embryogenic AGP composition. In an embodiment of this invention, the contacting occurs between about one week and about twelve weeks or about four weeks and about eight weeks. In an embodiment of this invention, the contacting first occurs for about four weeks, the contacting is transiently interrupted for transfer of the cell or tissue to fresh medium comprising an AGP composition effective for fostering somatic embryogenic competence, and contacting is resumed for about an additional four weeks. In an embodiment of this invention, this cycle is optionally performed repeatedly, e.g. contacting, transiently interrupting contacting, and secondly contacting. Contacting includes transient removal for repeating contacting with an AGP composition. AGP compositions effective for fostering somatic embryogenic competence include AGP compositions that are more effective when replaced after passage of a selected contacting time.

In an embodiment of this invention, the pro-embryogenic AGP composition is derived from the same species as the plant cell. In an embodiment of this invention, the plant cell or tissue and the source from which the AGP composition is originally derived are of the same plant variety. In an embodiment of this invention, the plant cell or tissue and the source from which the pro-embryogenic AGP composition is originally derived are not of the same plant variety. In an embodiment of this invention, the pro-embryogenic AGP composition is derived from a more embryogenic variety of the species compared to the variety of the plant cell. In an embodiment of this invention, the plant cell or tissue and the source from which the pro-embryogenic AGP composition is originally derived are not of the same plant species.

In an embodiment of this invention, the plant cell or tissue is not of a plant selected from the group consisting of: carrot, cucumber, spruce, chicory, tomato, cabbage, and Arabidopsis thaliana. In an embodiment of this invention, the plant cell or tissue is not a microspore. In an embodiment of this invention, the pro-embryogenic AGP composition is not from embryogenic callus of a plant selected from the group consisting of: carrot, cucumber, spruce, chicory, tomato, cabbage, Arabidopsis thaliana, and Acacia senegal. In an embodiment of this invention, the plant cell or tissue is not of a cotton variety selected from the group consisting of: Coker cotton varieties, Coker 201, Coker 310, Coker 315, Coker 320, Acala cotton varieties, Siokra 1-3, Siokra 1-4, Siokra S324, T25, GSA 25, GSA 71, GSA 75, G 8160, SJ-2, GSA 78, MCU-5, CNPA Precoce 2, Deltapine 90, GB-35B126, CRI12, DCH 32, CCRI12, Maxxa, Ultima, Riata, and Simian-3.

In an embodiment of this invention, the plant cell or tissue and/or the source of the embryogenic AGP composition is a dicot or a monocot. In an embodiment of this invention, the plant cell or tissue and/or the source of the AGP composition is of an agronomically useful plant. In an embodiment of this invention, the plant cell or tissue and/or the source of the pro-embryogenic AGP composition is of a fiber-producing plant. In an embodiment of this invention, the plant cell or tissue and/or the source of the pro-embryogenic AGP composition is of the Order Malvales. In an embodiment of this invention, the plant cell or tissue is of a species or variety that is recalcitrant to regeneration.

In an embodiment of this invention, the plant cell or tissue and/or the source of the AGP composition is a cotton cell or tissue. In an embodiment of this invention, the cotton cell or tissue and/or the source of the pro-embryogenic AGP composition is Upland cotton, Pima cotton, Egyptian cotton, Sea Island cotton, G. hirsutum, G. barbadense, tree cotton, Creole cotton, Levant cotton, Sturt's desert rose cotton, Thurber's cotton, or Hawaii cotton. In an embodiment of this invention, the cell or tissue is callus, hypocotyl, petiole, leaf, root, shoot, stem, transition region, cotyledon, stomata, anther, microspore, flower, primordium, or apex. In an embodiment of this invention, the plant cell or cells of the plant tissue have a cell wall. In an embodiment of this invention, the plant cell or tissue is not a protoplast.

In an embodiment of this invention, the cell or tissue is a callus cell or tissue and has been derived from callus, hypocotyl, petiole, leaf, root, shoot, stem, transition region, cotyledon, stomata, anther, microspore, flower, primordium, or an apex. In an embodiment of this invention, fostering somatic embryogenic competence also consists of the step of inducing formation of the callus cell or callus tissue from a hypocotyl, petiole, leaf, root, shoot, stem, transition region, cotyledon, stomatal, anther, microspore, flower, primordium, or apical cell. In an embodiment of this invention, the method for fostering somatic embryogenic competence also comprises inducing callus formation in the plant cell or tissue. In an embodiment of this invention, inducing callus formation occurs for about five weeks. In an embodiment of this invention, the contacting step occurs after or simultaneously with inducing callus formation.

In an embodiment of this invention, the plant cells or tissues are contacted at about 29-30° C. In an embodiment of this invention, the plant cells or tissues are exposed to a light intensity of about 5-15 μE (microEinsteins, micro-mols of photons per meter squared per second), with a photoperiod of 16 h.

In an embodiment of this invention, the plant cell or tissue is of a variety, cultivar, or line selected from the group consisting of Coker 315, Sicala 40, Siokra 1-4, Sicot 189, Emerald, Sicala 43, Sicala 45, Sicala V-2, Sicot 53, Sicot 70, Sicot 71, Sicot 80, Siokra S-102, Siokra V-16, Siokra V-17, Siokra V-18, Pearl, Sapphire, Topaz, Opal, Diamond, transgenic cotton varieties, Sicot 11B, Sicot 12B, Sicot 13B, Sicot 14B, PSC 355, 1517-77, 1517-95, 1517-99, Acala MAXXA, Acala Riata, Acala Sierra, AG 3601, Atlas, BXN 47, BXN 49B, DP 388, DP 422, DP 436, DP 449, DP 451, DP 458, DP 468, DP 5415, DP 555, DP 5690, DP 6207 Acala, DP 655, DP 655, FM 832, FM 958, FM 966, FM 989, FM 989, FM 991, HS 44, NuCOTN 33, NuCOTN 35, Paymaster HS 26, PHY 72 Acala, PHY 78 Acala, PM 1199, PM 1218, PM 1560, PM 2145, PM 2156, PM 2167, PM 2200, PM 2266, PM 2280, PM 2326, PM 2379, SG 215, ST 2454, ST 457, ST 4691, ST 4793, ST 4892, ST 4892, ST 5303, ST 5599, SG 105, SG 125, SG 501, SG 521, Xpress, a variety in the National Cotton Germplasm Collection (Germplasm Resources Information Network—GRIN, Cotton Collection, Curator: Percival, A. E. (713-260-9311), USDA-ARS, Texas A&M University, P.O. Box DN—Cotton Genetics Research, College Station, Tex. 77841), or a variety made by crossing one of the above-mentioned varieties. Cotton varieties useful in the practice of this invention include historical cotton varieties, elite cotton varieties, and as yet to be invented cotton varieties.

In an embodiment of this invention, the pro-embryogenic AGP composition is derived from a cotton variety selected from the group consisting of: Coker 315, Siokra 1-4, and Sicala 40.

In an embodiment of this invention, the pro-embryogenic AGP composition is derived from embryogenic callus, proembryonic masses, and/or embryos, or media that has been in contact with the above-mentioned cells and tissues. In an embodiment, the pro-embryogenic AGP composition is not derived from media. Embryos useful in the practice of this invention include zygotic and somatic embryos. In an embodiment of this invention, the pro-embryogenic AGP composition is not derived from zygotic embryos, seeds, or seed pods. In an embodiment of this invention, the pro-embryogenic AGP composition is derived from a plant gum.

In an embodiment of this invention, the pro-embryogenic AGP composition comprises a final concentration in the callus culture medium of between about 0.01 mg/L of medium and about 100 mg/L of medium. In an embodiment of this invention, the pro-embryogenic AGP composition comprises a concentration of between about 0.05 mg/L of medium and about 50 mg/L of medium. In an embodiment of this invention, the pro-embryogenic AGP composition comprises a concentration of between about 0.08 mg/L of medium and about 30 mg/L of medium. In an embodiment of this invention, the pro-embryogenic AGP composition comprises a concentration of between about 0.1 mg/L of medium and about 20 mg/L of medium. In an embodiment of this invention, the pro-embryogenic AGP composition comprises a concentration of between about 0.5 mg/L of medium and about 10 mg/L of medium. In an embodiment of this invention, the pro-embryogenic AGP composition comprises a concentration of between about 1 mg/L of medium and about 4 mg/L of medium. In an embodiment of this invention, the pro-embryogenic AGP composition comprises a concentration of between about 1 mg/L of medium and about 2 mg/L of medium.

In an embodiment of this invention, the pro-embryogenic AGP composition comprises purified AGP. In an embodiment of this invention, the embryogenic AGP is purified by Yariv reagent extraction. In an embodiment of this invention, the pro-embryogenic AGP is total AGP. In an embodiment of this invention, the pro-embryogenic AGP is further purified or fractionated by a hydropathic separation methodology. In an embodiment of this invention, the pro-embryogenic AGP is further purified by reverse phase high performance liquid chromatography (RP-HPLC), a hydropathic separation methodology. In an embodiment of this invention, the pro-embryogenic AGP composition is not purified using an antibody.

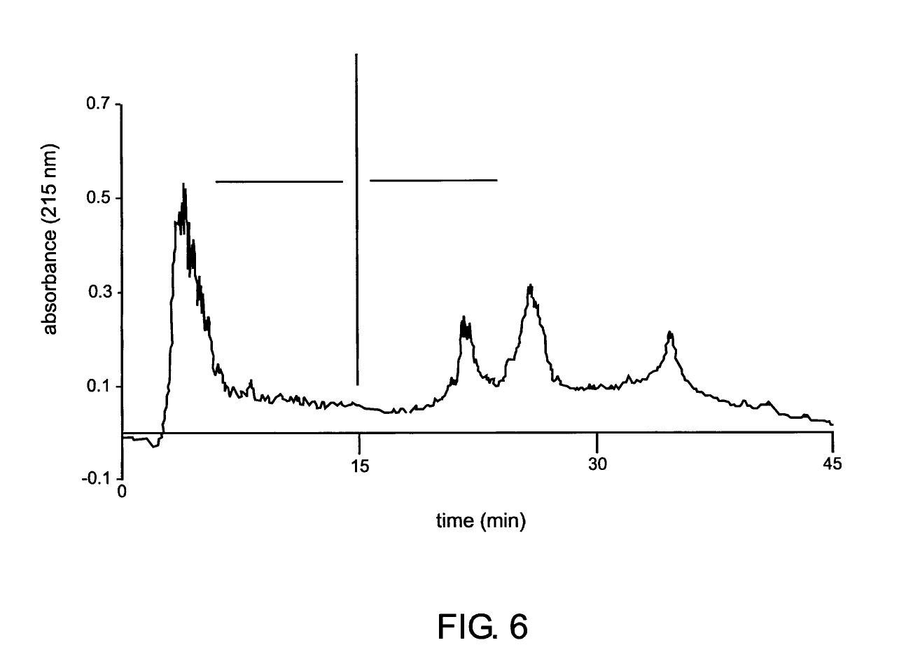

In an embodiment of this invention, the pro-embryogenic AGP composition comprises a hydrophobic pro-embryogenic AGP fraction. In an embodiment of this invention, the hydrophobic pro-embryogenic AGP fraction comprises AGP that elutes from a Brownlee Aquapore OD-300μ 7 m reverse-phase HPLC column (2.1×100 mm) (Perkin Elmer, Wellesley, Mass., USA) that has been equilibrated in 0.1% v/v trifluoroacetic acid (TFA), using a linear gradient from 0% acetonitrile and 0.1% v/v TFA to 80% v/v acetonitrile, 0.089% v/v TFA over 60 min at a flow rate of 0.5 mL/min, as described in Example 3 and shown in FIG. 6. Alternatively, AGP fractionation can be carried out using a semi-preparative Zorbax 300 SB-C8 9.4 mm×25 cm column eluted with a gradient of from about 20% to about 80% acetonitrile at a flow rate of 3 mL/min., as described in Example 9 and shown in FIG. 6. A range of elution times or acetonitrile concentrations are useful for separating the hydrophilic and hydrophobic peaks, in the bimodal distribution, from each other, but it is preferable to minimize the amount of the hydrophilic peak tail in the hydrophobic fraction.

In an embodiment of this invention, the pro-embryogenic AGP composition comprises the hydrophobic embryogenic AGP fraction at a concentration of between about 0.0015 mg/L of medium and about 15 mg/L of medium. In an embodiment of this invention, the pro-embryogenic AGP composition comprises a concentration of between about 0.0075 mg/L of medium and about 7.5 mg/L of medium. In an embodiment of this invention, the pro-embryogenic AGP composition comprises a concentration of between about 0.012 mg/L of medium and about 4.5 mg/L of medium. In an embodiment of this invention, the pro-embryogenic AGP composition comprises a concentration of between about 0.015 mg/L of medium and about 3 mg/L of medium. In an embodiment of this invention, the pro-embryogenic AGP composition comprises a concentration of between about 0.075 mg/L of medium and about 1.5 mg/L of medium. In an embodiment of this invention, the AGP composition comprises a concentration of between about 0.15 mg/L of medium and about 0.6 mg/L of medium. In an embodiment of this invention, the pro-embryogenic AGP composition comprises a concentration of between about 0.15 mg/L of medium and about 0.3 mg/L of medium.

In an embodiment of this invention, the pro-embryogenic AGP composition comprises hydrophobic peak #1, as shown in FIG. 8. In an embodiment of this invention, the hydrophobic pro-embryogenic AGP fraction comprises AGP that elutes using the materials and methods described above, at about 27% to about 32% acetonitrile, as shown in FIG. 8. In an embodiment of this invention, the pro-embryogenic AGP composition consists essentially of hydrophobic peak #1.

In an embodiment of this invention, the pro-embryogenic AGP composition comprises hydrophobic peak #1 at a concentration of between about 0.0008 mg/L of medium and about 8 mg/L of medium. In an embodiment of this invention, the pro-embryogenic AGP composition comprises hydrophobic peak #1 at a concentration of between about 0.004 mg/L of medium and about 4 mg/L of medium. In an embodiment of this invention, the pro-embryogenic AGP composition comprises hydrophobic peak #1 at a concentration of between about 0.0064 mg/L of medium and about 2.4 mg/L of medium. In an embodiment of this invention, the pro-embryogenic AGP composition comprises hydrophobic peak #1 at a concentration of between about 0.008 mg/L of medium and about 1.6 mg/L of medium. In an embodiment of this invention, the pro-embryogenic AGP composition comprises hydrophobic peak #1 at a concentration of between about 0.04 mg/L of medium and about 0.8 mg/L of medium. In an embodiment of this invention, the pro-embryogenic AGP composition comprises hydrophobic peak #1 at a concentration of about 0.008 mg/L of medium and about 0.32 mg/L of medium. In an embodiment of this invention, the pro-embryogenic AGP composition comprises hydrophobic peak #1 at a concentration of between about 0.008 mg/L of medium and about 0.16 mg/L of medium.

In an embodiment of this invention, the pro-embryogenic AGP composition comprises hydrophobic peak #1 or #2, such as shown in FIG. 8, eluting using the materials and methods described above, at about 27% and about 32% acetonitrile and between about 32% and about 37% acetonitrile, respectively. In an embodiment of this invention, the pro-embryogenic AGP composition comprises a fraction susceptible to proteolytic, including tryptic, cleavage.

In the practice of this invention, when harvesting or purifying an AGP fraction or peak, the bounds of the fraction or peak, in time units and/or elution buffer composition, are selected to harvest or purify the selected fraction or peak at a selected purity relative to contamination by other fractions or peaks that can be harvested or purified by the selected method. It is preferable to select bounds that do not compromise the activity of the selected fraction or peak. In an embodiment of this invention, when harvesting a hydrophobic embryogenic AGP fraction, the lower hydrophobicity bound is between about 7.5 minutes and about 18 minutes when utilizing the RP-HPLC protocol in Example 3.

AGP compositions useful in the practice of this invention include unextracted and unpurified AGP including cell lysate, Yariv reagent extracted AGP, total AGP, purified AGP, fractionated AGP, chitinase treated AGP, deglycosylated AGP, dearabinosylated AGP, deglycosylated and dearabinosylated AGP, AGP with and without post-translational modification, hydrophobic AGP fractions, hydrophobic AGP peaks #1 and #2, protease treated AGP, AGP peptide fragments, engineered AGP that is arabinosylated and/or glycosylated, AGP that is differently arabinosylated and/or glycosylated, engineered AGP that is not arabinosylated and/or glycosylated, and chemically synthesized AGP. Each engineered AGPs is derived from an original source from which an AGP amino acid or DNA sequence was utilized to design the engineered AGP. Engineered AGPs useful in the practice of this invention optionally contain additional domains and/or sequences, as is known art.

In an embodiment of this invention, the percentage of embryogenic explants is increased by at least about 20%, at least about 50%, at least about 75%, or at least about 100%, as compared to explants that have not been contacted by embryogenic AGP.

In an embodiment of this invention, contacting the cell or tissue with an AGP composition effective for fostering somatic embryogenic competence decreases the time until the plant cell or tissue undergoes somatic embryogenesis by about two weeks, by at least 25%, or by at least 50% relative to not contacting with an AGP composition.

In an embodiment of this invention, the plant cell or tissue is in culture, e.g., in vitro, on solid medium, or in a suspension culture. In an embodiment of this invention, the plant cell or tissue is in vivo, e.g., the plant having the cell or tissue is grown in soil in non-sterile conditions. In an embodiment of this invention, the plant is wounded before being contacted with an AGP composition.

In an embodiment of this invention, the plant cell or tissue is in culture in contact with culture medium having no hormones or having hormones selected from the group consisting of hormone cocktail A, B, C, D, or E. In an embodiment of this invention, the culture medium contains about 0.5 mg/L of medium kinetin and about 1 mg/L of medium indole-3-butyric acid, hormone cocktail D. In an embodiment of this invention, the culture medium contains about twice the concentration of indole-3-butyric acid as kinetin.

In an embodiment of this invention, the culture medium contains purified AGP, embryogenic callus AGP, total AGP, the hydrophobic AGP fraction, hydrophobic peak #1, hydrophobic peak #2, deglycosylated AGP, dearabinosylated AGP, deglycosylated and dearabinosylated AGP, chitinase treated AGP, or mixtures thereof. In an embodiment of this invention, the deglycosylated AGP is about 26 kD.

This invention provides somatic embryos produced by the method for fostering somatic embryogenic competence. This invention provides somatic embryogenic callus and/or somatic embryos produced by the method for fostering somatic embryogenic competence.

This invention provides a method for regenerating a plant comprising harvesting a plant cell or tissue from a first plant; contacting the plant cell or tissue with an AGP composition effective for fostering somatic embryogenic competence; and regenerating a second plant from the plant cell or tissue. This invention provides plants and progeny produced by the above-described method. This invention provides seeds produced by the above-described plants and progeny.

This invention provides a method for transforming a plant comprising: harvesting a plant cell or tissue from a plant; transforming the plant cell or tissue; contacting the transformed plant cell or tissue with an AGP composition effective for fostering somatic embryogenic competence; and regenerating a transformed plant from said plant cell or tissue. This invention provides a method for transforming Siokra 1-4. This invention provides transformed plants and progeny produced by the above-described method. This invention provides seeds produced by the above-described transformed plants and progeny.

This invention provides a method for making an AGP composition useful for fostering somatic embryogenic competence comprising: providing embryogenic callus; and harvesting pro-embryogenic AGP from said embryogenic callus. In an embodiment of this invention, the harvesting comprises Yariv extraction and/or hydropathic fractionation (e.g. RP-HPLC). In an embodiment of this invention, the harvesting also comprises collecting RP-HPLC the hydrophobic fraction, hydrophobic peak #1, and/or hydrophobic peak #2.

This invention provides a method for making an AGP composition useful for fostering somatic embryogenic competence comprising: expressing a protein or peptide comprising an amino acid sequence selected from the group consisting of SEQ ID NOS:1-7, 15 and 17; and harvesting the protein or peptide. This invention provides a method for making an AGP composition useful for fostering somatic embryogenic competence comprising: expressing a protein comprising a peptide having a sequence selected from the group consisting of SEQ ID NOS:1-7, and SEQ ID NOS:15 and 17 and SEQ ID NOS: 25 and 26 and tryptic digests thereof, and harvesting the protein or peptide. In an embodiment of this invention, the protein or peptide is expressed in a plant host or a non-plant host, as is known in the art.

This invention provides a method for making a plant cell culture medium effective for fostering somatic embryogenic competence comprising: providing a plant cell culture medium and adding an AGP composition effective for fostering somatic embryogenic competence.

This invention provides a method for impeding somatic embryogenic competence in a plant cell or tissue comprising contacting the plant cell or tissue with an AGP composition effective for impeding somatic embryogenic competence, compared to not contacting the plant cell or tissue with the AGP. In an embodiment of this invention, somatic embryogenesis is impeded by at least about 10%, 50%, or 90%.

In an embodiment of the method for impeding somatic embryogenic competence, the AGP composition comprises total AGP from non-embryogenic callus, the hydrophilic AGP, hydrophilic peak #1, AGP derived from a variety that is less embryogenic than the plant cell or tissue, or mixtures thereof. In an embodiment of this invention, the AGP composition consists essentially of the hydrophilic fraction, hydrophilic peak #1, or mixtures thereof.

This invention provides a method for maintaining a plant cell or tissue in culture comprising contacting the plant cell or tissue with an AGP composition effective for plant cell or tissue maintenance. In an embodiment of this invention, the plant cell or tissue is maintained for about 25% to about 100% longer compared to not contacting.

This invention provides a method for fostering callus formation in a plant cell or tissue comprising contacting said plant cell with an AGP composition effective for fostering callus formation.

This invention provides a method for culturing a plant cell comprising contacting said plant cell with a culture medium comprising about 0.5 mg/L of medium kinetin and about 1 mg/L of medium indole-3-butyric acid.

This invention provides a method for fostering somatic embryogenic competence in a plant cell or tissue comprising contacting said plant cell with a composition comprising 0.5 mg/L of medium kinetin and 1 mg/L of medium indole-3-butyric acid.

This invention provides a purified pro-embryogenic AGP composition effective for fostering somatic embryogenic competence of a plant cell or tissue. In an embodiment of this invention, the pro-embryogenic AGP composition is derived from embryogenic callus, proembryonic masses, and/or somatic embryos, including embryogenic callus that was generated using hormones. In an embodiment of this invention, the AGP composition is derived from a dicot, a monocot, an agronomically useful plant, a fiber-producing plant, a Malvales, or cotton. In an embodiment of this invention, the AGP composition is derived from a cotton variety selected from the group consisting of Coker varieties, Coker 315, Siokra 1-4, and Sicala 40.

In an embodiment of this invention, the pro-embryogenic AGP composition effective for fostering somatic embryogenic competence comprises AGP from embryogenic callus, total AGP, a hydrophobic AGP fraction, hydrophobic peak #1, hydrophobic peak #2, or mixtures thereof. In an embodiment of this invention, when AGP compositions consist essentially of a fraction, peak, or a mixture thereof, useful concentrations of the fraction, peak, or mixture thereof, are determined using experimental data on the effectiveness of total AGP from the same source and the proportion of the fraction, peak, or mixture in the total AGP.

In an embodiment of this invention, the embryogenic AGP composition comprises total AGP at a concentration of between about 0.01 mg/L of medium and about 100 mg/L of medium. In an embodiment of this invention, the pro-embryogenic AGP composition comprises a concentration of between about 0.05 mg/L of medium and about 50 mg/L of medium. In an embodiment of this invention, the pro-embryogenic AGP composition comprises a concentration of between about 0.08 mg/L of medium and about 30 mg/L of medium. In an embodiment of this invention, the pro-embryogenic AGP composition comprises a concentration of between about 0.1 mg/L of medium and about 20 mg/L of medium. In an embodiment of this invention, the pro-embryogenic AGP composition comprises a concentration of between about 0.5 mg/L of medium and about 10 mg/L of medium. In an embodiment of this invention, the pro-embryogenic AGP composition comprises a concentration of between about 1 mg/L of medium and about 4 mg/L of medium. In an embodiment of this invention, the pro-embryogenic AGP composition comprises a concentration of between about 1 mg/L of medium and about 2 mg/L of medium.

In an embodiment of this invention, the pro-embryogenic AGP composition comprises the hydrophobic fraction at a concentration that is at the same as the concentration of the hydrophobic AGP fraction in the above-listed total AGP concentrations. In an embodiment of this invention, the hydrophobic fraction is 15%-25% of total AGP. This invention provides a composition effective for fostering somatic embryogenic competence comprising 0.15 mg/L of medium hydrophobic AGP fraction. In an embodiment of this invention, the embryogenic AGP composition comprises hydrophobic peak #1 at a concentration that is at the same as the concentration of hydrophobic peak #1 in the above-listed total AGP concentrations. In an embodiment of this invention, hydrophobic peak #1 is 4% of total AGP. This invention provides a composition effective for fostering somatic embryogenic competence comprising 0.08 mg/L of medium hydrophobic peak #1.

In an embodiment of this invention, the plant culture medium is provided as a dry or concentrated composition to which water is to be added.

In an embodiment of this invention, AGP is harvested from embryogenic callus that has been in contact with an AGP composition effective for fostering somatic embryogenic competence of a plant cell or tissue. In an embodiment of this invention, somatic embryos visible to the human eye are optionally removed from the embryogenic callus prior to harvesting the AGP. In an embodiment of this invention, the removed somatic embryos are optionally regenerated into plants.

In an embodiment of this invention, the AGP composition comprises a protein or peptide having a sequence of SEQ ID NOS: 15, 17, 25 or 26, capable of being encoded by SEQ ID NOS: 14 or 16, of a portion or at least about fifteen amino acids of SEQ ID NOS:15 or 17, or having 80% sequence similarity to SEQ ID NOS:15 or 17 or a tryptic digest of SEQ ID NOS: 25 or 26. In an embodiment of this invention, the AGP composition has a sequence of a phytocyanin-like domain (e.g., proteins PL-1 (SEQ ID NO:25) or PL-2 (SEQ ID NO:26) or a tryptic digest thereof) or a pro-rich domain (e.g., amino acids 139-156 of SEQ ID NO:15 and amino acids 131-182 of SEQ ID NO:17). In an embodiment of this invention, the protein or peptide is optionally engineered, not arabinosylated and/or glycosylated, differently arabinosylated and/or glycosylated than the AGP from which it was derived, and/or chemically synthesized.

In an embodiment of this invention, the AGP composition comprises a peptide having a sequence of SEQ ID NOS: 1-7. In an embodiment of this invention, the peptide is optionally engineered, not arabinosylated and/or glycosylated, differently arabinosylated and/or glycosylated than the AGP from which it was derived, and/or chemically synthesized.

In the practice of this invention, such as when using plants other than cotton or tissues other than embryogenic callus for harvesting AGP, it is useful to determine what concentrations of AGP are effective for fostering somatic embryogenic competence and/or what fraction(s) contains AGP effective for fostering or impeding somatic embryogenic competence. Determining what concentrations or fractions are useful in for practicing any of the methods of this invention, can be performed by methods known to the art and provided by this invention, without undue experimentation. Active and inactive fractions are determined empirically, depending on what fractionation method is utilized. It will be understood in the art that the AGPs of other plant species or varieties, or other plant tissues, can show different fractionation patterns or may be more effective if fractionated by different methods. Pro-embryogenic fractions can be identified by experiments similar to those described in the examples below.

In the practice of this invention, after seeds are harvested and ginned (removal of lint) they are rested, preferably for at least a month, before being germinated. As used herein, “freshly harvested” seeds are seeds that have been rested for no more than about one year.

Methods are known in the art for Agrobacterium mediated plant transformation (Gelvin, S. B., (March 2003) Microbiology and Molecular Biology Reviews 67(1):16-37; Gould, J. H., (1998) Plant Molecular Biology Reporter 16(3):284-289; Sunilkumar, G. et al., (August 2001) Molecular Breeding, 8(1):37-52; Wilkins, T. A. et al., (1998) Cotton Biotechnology Workshop (Beltwide Cotton Conference) San Diego, Calif.; and Satyavathy, V. V. et al., (February 2002) Plant Science 162(2):215-223).

Methods are known in the art for distinguishing embryogenic from non-embryogenic callus. Embryogenic callus is brownish in color ranging from light to dark and can include gray, gray-green and yellow. It is drier, more friable and more granular than non-embryogenic callus, which is generally greener, softer and wetter. (Patterson, A. H. and Smith R. H. (1999) “Future Horizons, Biotechnology for Cotton Improvement” in Cotton. Origin, History, Technology and Production, C. W. Smith and J. T. Cothren eds. John Wiley & Sons, New York, pg. 415).

Methods for harvesting embryogenic callus useful in the practice of this invention include: harvesting an entire explant, harvesting embryogenic callus and embryos, and harvesting embryogenic callus with embryos removed.

RP-HPLC methods known in the art are useful in the practice of this invention. In an embodiment, both equilibration buffer and elution buffer do not denature selected AGPs. Techniques useful for AGP fractionation include: hydropathic fractionation, antibody precipitation, antibody chromatography, ion-exchange chromatography, electrophoresis, size-exclusion chromatography, methods known in the art for separating peptides and proteins, and methods yet to be discovered for separating peptides and proteins.

Methods for culturing or growing embryogenic callus known in the art are useful in the practice of this invention. In an embodiment of this invention, callus tissue is cultured for longer than about eight weeks. In practice, culturing callus tissue longer than about eight weeks does not appear to increase the percent of embryogenic explants, but the number of embryos and amount of embryogenic callus is increased.

A protein is considered an isolated protein if it is a protein purified at least two-fold from a host cell or culture medium in which it naturally occurs or is recombinantly produced. It can be purified or it can simply be substantially free of other proteins and biological materials with which it is associated in nature.

An isolated nucleic acid is a nucleic acid outside of the context in which it is found in nature. An isolated nucleic acid is a nucleic acid having a structure that is not identical to the entirety of any naturally occurring nucleic acid molecule. The term covers, for example: (a) a DNA which has the sequence of part of a naturally-occurring genomic DNA molecule, but is not flanked by both of the coding or noncoding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally-occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein, or a modified gene having a sequence not found in nature.

DNA constructs prepared for introduction into a prokaryotic or eukaryotic host will typically comprise a replication system (i.e. vector) recognized by the host, including the intended DNA fragment encoding the desired polypeptide, and will preferably also include transcription and translational initiation regulatory sequences operably linked to the polypeptide-encoding segment. Expression systems (expression vectors) may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross cell membranes or be secreted from the cell. Sequences useful for isolation of the encoded protein may also be included.

Mutational, insertional, and deletional variants of the disclosed nucleotide sequences can be readily prepared by methods which are well known to those skilled in the art. These variants can be used in the same manner as the exemplified primer sequences so long as the variants have substantial sequence homology with the original sequence. It is well known in the art that the polynucleotide sequences of the present invention can be truncated and/or mutated such that certain of the resulting fragments and/or mutants of the original full-length sequence can retain the desired characteristics of the full-length sequence. See, for example, Maniatis (1982) Molecular Cloning: A Laboratory Manual, Cold Spring Harbor Laboratory, New York, pages 135-139, incorporated herein by reference.

### EXAMPLES

### Example 1

### Growth of Embryogenic and Non-Embryogenic Callus

Hypocotyl explants from cotton variety Coker 315 were grown on basic media with hormones to initiate (induce) callus. Basic media contained:
1× Murashige and Skoog salt mixture (cat. no. 11117-074, Invitrogen Corporation, Carlsbad, Calif., USA)
1× Gamborg's vitamin solution (cat. no. G1019, Sigma, St. Louis, Mo., USA)
30 g/L glucose
1.9 g/L potassium nitrate
0.9 g/L magnesium chloride hexahydrate
0.1 g/L myo-inositol
2 g/L gellan gum
pH 5.8
   Hormones were added:
0.1 mg/L kinetin
0.1 mg/L 2,4-dichlorophenoxyacetic acid (2,4-D)

1-2 cm segments of hypocotyls from 10 day-old dark-grown seedlings were grown on basic media at 29° C. with a 16 h photoperiod and a light intensity of 5-15 μE (microEinsteins, micro-mols of photons per meter squared per second) for 5 weeks. They were then transferred to basic media without hormones kinetin and 2,4-D. The callus was transferred to fresh basic media without hormones every 4 weeks. Embryogenic and non-embryogenic callus were both successfully grown.

### Example 2

### Extraction of Total AGP from Embryogenic and Non-Embryogenic Callus

After 5 weeks on medium #1, with added hormones, followed by 12 weeks on medium #1 without hormones, tissue was harvested from the embryogenic and non-embryogenic callus grown in Example 1. AGPs were extracted separately from the embryogenic and non embryogenic callus. Embryogenic AGP has also been extracted from embryogenic callus produced without hormones, for example harvested after 13 weeks on medium #1 without hormones.

AGPs were extracted using a modification of the protocol in Gane A M, et al. (1995) Carbohydr Res. 277:67-85.

Lyophilized cotton callus was ground to a fine powder in liquid nitrogen. Soluble components were extracted at 4° C. for ˜3 h in 50 mM Tris HCl, pH 8.0, containing 10 mM EDTA, 1% Triton X-100 and 0.1% β-mercaptoethanol (40 mL buffer per g lyophilized tissue). The extract was then centrifuged (16,300 g, 1 h) and the supernatant retained. High molecular weight components were then precipitated overnight at −20° C. by adding 5 volumes ethanol. The precipitate was centrifuged (16,000 g, 20 min) and the supernatant discarded. The pellet was dried before it was resuspended in water and lyophilized. The lyophilized material was then dissolved in 1% w/v NaCl and an equal volume of a solution of 2 mg/mL β-glucosyl Yariv reagent in 1% w/v NaCl was added. The mixture was left to precipitate overnight at 4° C., and the insoluble β-glucosyl Yariv-AGP complex was collected by centrifugation (18,000 g, 1 h), washed twice with 1% w/v NaCl and twice with methanol. The pellet was dried, dissolved in a minimum of dimethyl sulfoxide, and solid sodium dithionite was added to ˜30% w/v. Water was added at an equal volume to dimethyl sulfoxide and the mixture was vortexed and incubated at room temperature for 5 min. The resultant clear yellow solution was centrifuged (18,000 g, 2 min) and the supernatant applied to a pre-packaged Sephadex G-25 M PD-10 column (Amersham Biosciences, Piscataway, N.J., USA) for desalting. Purified AGP was eluted with water and lyophilized.

This method differed from Gane et al. in the method step used to disrupt the Yariv-AGP complex: dimethyl sulfoxide (DMSO) was used in place of water. Also, the Yariv-AGP-sodium dithionite mixture was not purged with nitrogen, sealed and stirred. The resulting extracted composition was similar, but this extraction method was faster.

### Example 3

### Characterization of Total AGP Extracted from Embryogenic and Non-Embryogenic Callus

AGPs yields were more than two-fold greater for the embryogenic compared to the non embryogenic callus, over several repetitions. (Table 1)

TABLE 1
Total AGP Yield from Cotton Callus

| Plant | Variety | Tissue | Yield (mg/g dry tissue) |
|---|---|---|---|
| Cotton | Coker 315 | non-embryogenic callus | 3.4 |
| Cotton | Coker 315 | embryogenic callus | 8.8 +/− 0.6 |

The total AGPs from both embryogenic and the non embryogenic callus were separated according to their hydrophobicity by reverse-phase high performance liquid chromatography (RP-HPLC).

AGP samples from embryogenic and non-embryogenic cotton Coker 315 callus extracted in Example 2 were solubilized in water and applied to a Brownlee Aquapore OD-300 7 μm reverse-phase HPLC column (2.1×100 mm) (Perkin Elmer, Wellesley, Mass., USA) equilibrated in 0.1% v/v trifluoroacetic acid (TFA). Fractions were eluted using a linear gradient from 0% acetonitrile and 0.1% v/v TFA to 80% v/v acetonitrile, 0.089% v/v TFA over 60 min at a flow rate of 0.5 mL/min. The profile of the eluted AGPs was indicated by absorbance at 215 nm (FIG. 1). Absorbance at 215 nm was approximately but not directly indicative of the amount of total AGP, because the amount of protein and carbohydrate can vary significantly from one AGP to another, and the absorbance at 215 primarily measured protein content. The buffers utilized for RP-HPLC did not denature the AGPs.

Several differences were demonstrated between embryogenic (dashed) and non-embryogenic callus (solid). Firstly, the embryogenic AGPs resolved into a major hydrophilic peak and three more hydrophobic peaks, whereas the AGPs from the non-embryogenic callus had only one significant peak and a tail which were also hydrophilic, but the peak had a slightly different retention time from that of the embryogenic AGPs. The HPLC peak from the non-embryogenic AGPs eluted at a retention time of approximately 2-8 min, corresponding to an acetonitrile concentration of ˜3-11%. Each embryogenic peak also had a tail.

### Example 4

### Fostering Somatic Embryogenic Competence

Coker 315 hypocotyl explants were grown on hormone free basic media. Although callus could be initiated on basic media with hormones, the entire process was slower on basic media with hormones than on basic media without hormones (see below).

After about five weeks on hormone-free basic media, the explants, were transferred to basic media with or without added AGPs extracted from embryogenic callus, Example 2. The AGP concentration was 1 mg/L. The explants were transferred to fresh media every four weeks. At time intervals, the callus was scored for embryogenic callus formation. Methods for distinguishing between embryogenic callus and non embryogenic callus are known in the art. Embryogenic callus, from which embryos can arise, is drier and grainier in texture and generally lighter brown in color than non-embryogenic callus, which is generally greener, softer and wetter. Scoring for embryogenic callus formation was quantitative, reproducible and AGP concentration dose dependent.

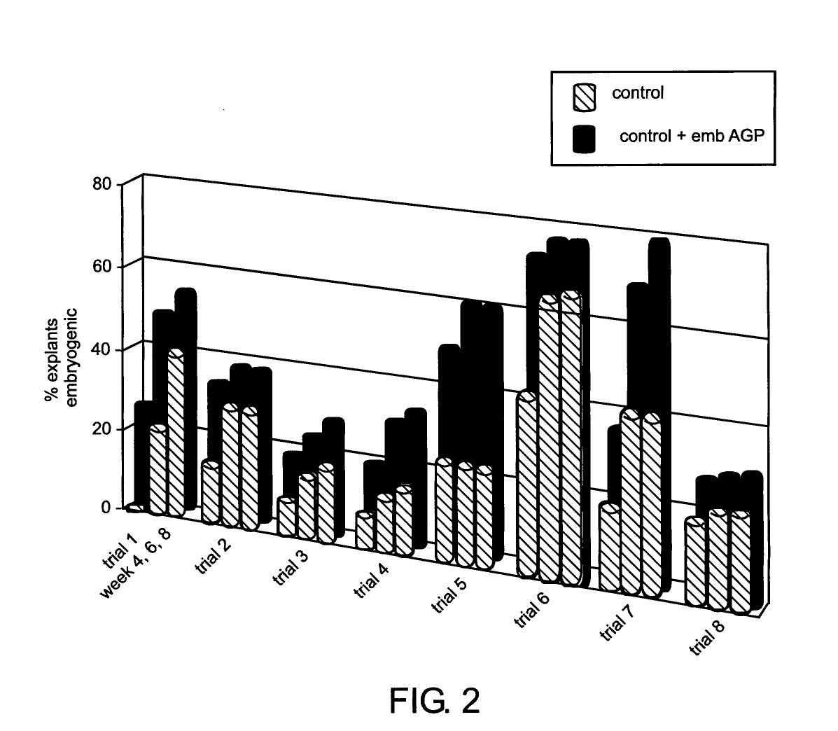

Table 2 and FIG. 2 show the percentage of explants having embryogenic callus in eight experiments. An explant was scored as being somatic embryogenesis competent when the explant was detected to have at least one section of embryogenic callus. The variation between experiments was determined to be due to differences in batches of seed from which the hypocotyls were produced and to differences in the position of the culture plates in the growth room which were exposed to slight temperature differences. Freshly harvested seed resulted in higher rates of production of embryogenic callus, as did higher temperatures. Preferably the temperature is 29-30° C. Preferably the intensity of light is 5-15 μE (microEinsteins, micro-mols of photons per meter squared per second). After seeds were harvested and ginned (removal of lint) they were rested, preferably for at least a month, before being germinated. Freshly harvested seeds have been rested for no more than about one year. Substantially more explants were embryogenic with AGPs extracted from embryogenic callus than without AGPs. 50-135 explants were scored for each trial. AGP fostered somatic embryogenic competence more effectively compared to the control during shorter contacting times.

TABLE 2
Fostering Somatic Embryogenic Competence by AGP

| | | week 4 + | | week 6 + | | week 8 + |
|---|---|---|---|---|---|---|
| | week 4 | emb | week 6 | emb | week 8 | emb |
| | control | AGP | control | AGP | control | AGP |
| trial | (%) | (%) | (%) | (%) | (%) | (%) |
| 1 | 0 | 25 | 21 | 48 | 40 | 54 |
| 2 | 14 | 33 | 29 | 37 | 29 | 37 |
| 3 | 8 | 18 | 15 | 23 | 18 | 28 |
| 4 | 8 | 19 | 13 | 29 | 16 | 33 |
| 5 | 24 | 49 | 24 | 60 | 24 | 60 |
| 6 | 43 | 73 | 65 | 77 | 67 | 77 |
| 7 | 19 | 36 | 42 | 69 | 59 | 79 |
| 8 | 19 | 27 | 22 | 29 | 22 | 30 |

As described above, callus can also be initiated on basic media with hormones, however the entire process was slower with hormones than on basic media without hormones. When grown on hormones, callus initially forms more quickly, however, this callus had to be transferred several times (about every 4 weeks) onto a hormone-free medium for the callus to become embryogenic. The earliest embryogenic callus formed with hormone in the basic medium was about 8 weeks after being transferred onto a hormone-free medium, but often it required more than 12 weeks. In contrast, embryogenic callus grown in the absence of hormones was formed about 4 weeks after the first transfer.

### Example 5

### Regeneration of Plants from AGP Induced Embryos

Embryos were selected from AGP fostered somatic embryogenic competence in Example 4, and regenerated into two fertile cotton plants from which viable seed was collected. No phenotypic differences were observed in the regenerated plants compared to the parent Coker 315 variety.

### Example 6

### Impeding Somatic Embryogenic Competence

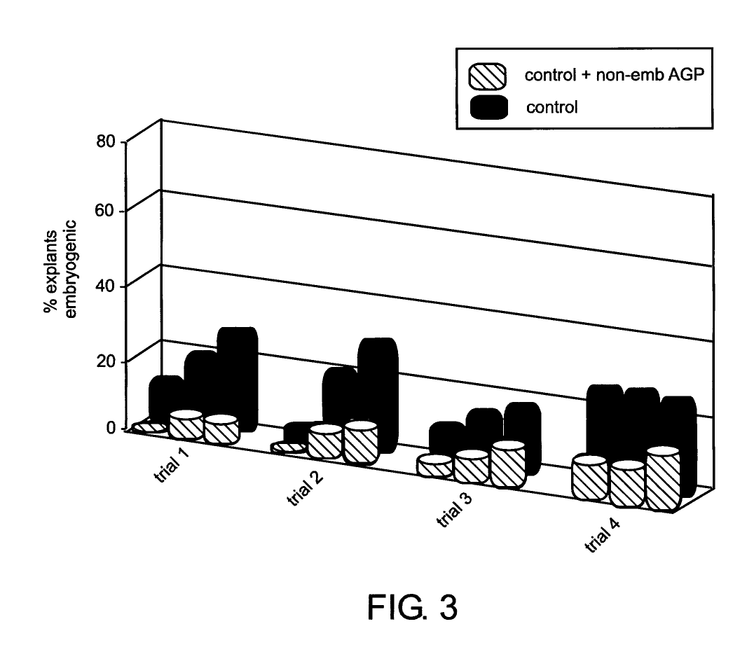

Coker 315 hypocotyls were grown on hormone free media (basic media). After the callus was established, the tissue samples containing callus, were transferred to basic media with or without added AGPs extracted from non-embryogenic callus, Example 2. The concentrations of AGP were 1 mg/L. The explants were transferred to fresh media every four weeks. At time intervals, the callus was scored for embryogenic callus formation. Table 3 and FIG. 3 show the percent of lines embryogenic in four experiments. Somatic embryogenesis was significantly impeded. Between 93-108 explants were scored for each trial.

TABLE 3
Impeding Somatic Embryogenic Competence by AGP

| | | non-emb | | non-emb | | non-emb |
|---|---|---|---|---|---|---|
| | control | AGP | control | AGP | control | AGP |
| | week 4 | week 4 | week 6 | week 6 | week 8 | week 8 |
| trial | (%) | (%) | (%) | (%) | (%) | (%) |
| 1 | 10 | 0 | 18 | 5 | 26 | 4 |
| 2 | 2 | 0 | 19 | 7 | 28 | 10 |
| 3 | 8 | 3 | 13 | 8 | 13 | 8 |
| 4 | 24 | 8 | 10 | 9 | 24 | 14 |

### Example 7

### Gum Arabic AGP has No Effect on Somatic Embryogenesis

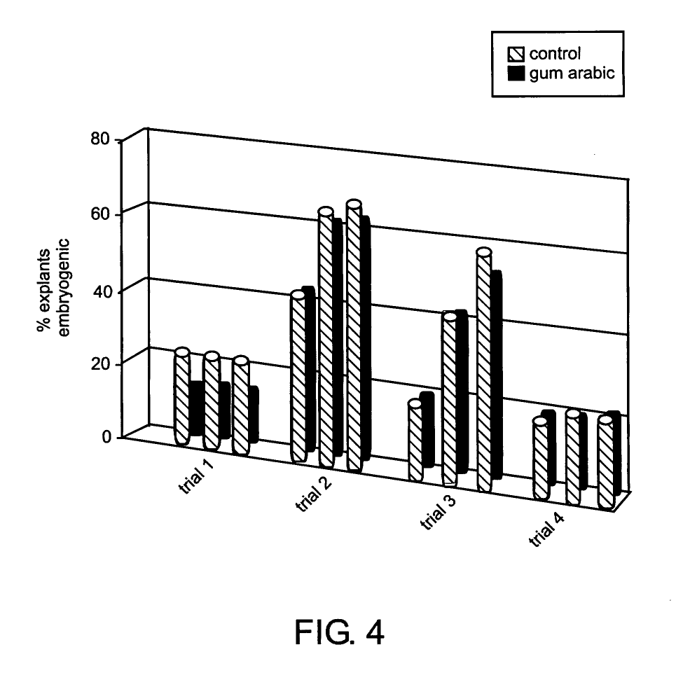

Coker 315 hypocotyls were grown on hormone free media (basic media). After the callus was established, the explants, were transferred to basic media with or without added gum Arabic AGPs, gum Arabic is an exudate from Acacia Senegal that is primarily AGP obtainable, e.g., from Sigma, St. Louis, Mo., USA. “AGPs from gum Arabic were Yariv-precipitated and subsequently treated in the same way as the previously described AGP purification. The concentration of gum arabic AGP utilized was 1 mg/L.” The explants were transferred to fresh media every four weeks. At time intervals, the callus was scored for embryogenic callus formation. Data is shown in Table 5 of the percent of embryogenic explants. Between 93-108 explants were scored for each trial. Table 4 and FIG. 4 show the results of four experiments. Between 71-135 explants were scored for each trial. Gum Arabic has no somatic embryogenic competence fostering activity and appeared to impede somatic embryogenic competence slightly.

TABLE 4
AGP
Gum Arabic Has No Activity

| | | Gum | | | | |
|---|---|---|---|---|---|---|
| | | Arabic | | Gum Arabic | | Gum Arabic |
| | control | AGP | control | AGP | control | AGP |
| | week 4 | week 4 | week 6 | week 6 | week 8 | week 8 |
| Trial | (%) | (%) | (%) | (%) | (%) | (%) |
| 1 | 24 | 13 | 24 | 14 | 24 | 14 |
| 2 | 43 | 43 | 65 | 61 | 67 | 62 |
| 3 | 19 | 19 | 42 | 41 | 59 | 52 |
| 4 | 19 | 19 | 22 | 19 | 22 | 20 |

### Example 8

### Fostering Somatic Embryogenic Competence Using Varying Concentrations of AGP

Coker 315 hypocotyls were grown on basic media (no hormones). After the callus was established, explants were transferred to basic media with or without added AGPs extracted from embryogenic callus, Example 2. The concentrations of AGP were between 1 mg/L and 4 mg/L. The explants were transferred to fresh media every four weeks. At time intervals, the callus was scored for embryogenic callus formation.

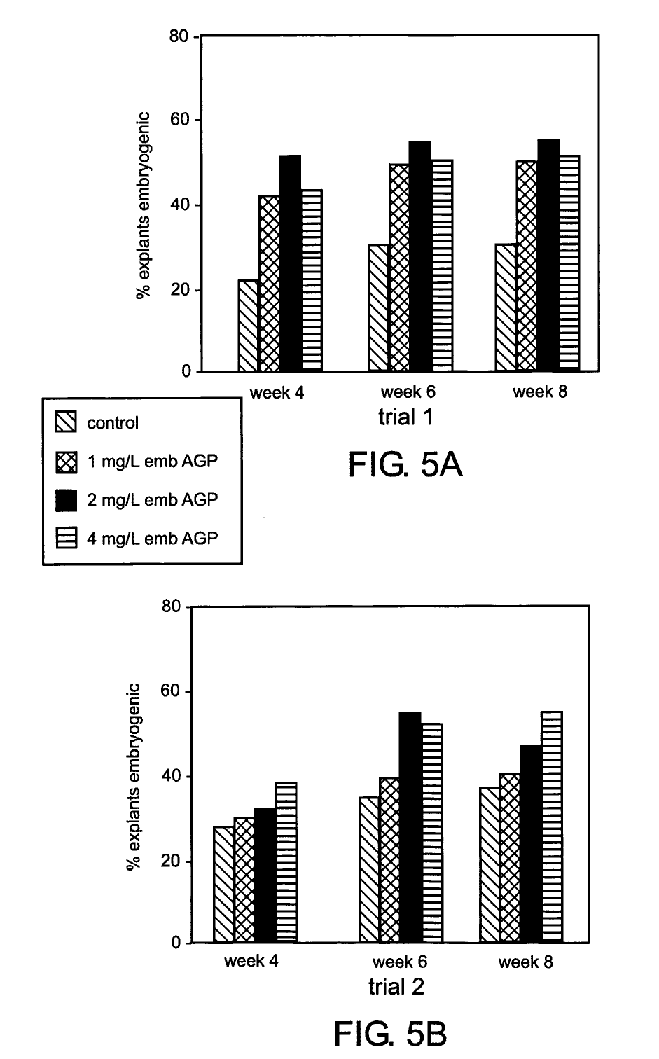

Table 5 shows the results of five experiments and FIGS. 5A and 5B show the results of two experiments of the percent of embryogenic explants. Between 28 and 120 explants were scored for each trial. Somatic embryogenic competence was fostered between about 40% and about 60% with about 1-2 mg/L AGP. In the first trial, the response flattened out between 2 and 4 mg/L. In the second and fifth trials, the response improved with increasing amounts of embryogenic AGP.

TABLE 5
Varying Concentration of Embryogenic AGP

| | | Control | | 2 mg/L AGP | 4 mg/L AGP |
|---|---|---|---|---|---|
| Trial | Week | (%) | 1 mg/L AGP (%) | (%) | (%) |
| 1 | 4 | 22 | 42 | 51 | 43 |
| 1 | 6 | 30 | 49 | 54 | 50 |
| 1 | 8 | 30 | 49 | 54 | 50 |
| 2 | 4 | 28 | 30 | 32 | 38 |
| 2 | 6 | 35 | 39 | 45 | 52 |
| 2 | 8 | 37 | 40 | 46 | 54 |
| 3 | 4 | 40 | 60 | 48 | 43 |
| 3 | 6 | 72 | 91 | 89 | 95 |
| 3 | 8 | 80 | 97 | 93 | 95 |
| 4 | 4 | 4 | 4 | 9 | 5 |
| 4 | 6 | 8 | 11 | 19 | 16 |
| 4 | 8 | 9 | 12 | 21 | 19 |
| 5 | 4 | 36 | 47 | 59 | 59 |
| 5 | 6 | 49 | 57 | 71 | 71 |
| 5 | 8 | 57 | 63 | 72 | 75 |

### Example 9

### Fractionation of Embryogenic AGPs

The total embryogenic AGPs extracted in Example 2 were split into the hydrophilic and hydrophobic fractions by RP-HPLC as in Example 3, but using a semi-preparative Zorbax 300 SB-C8 9.4 mm×25 cm column and a flow rate of 3 mL/min. The embryogenic AGP peaks appeared in a bimodal distribution. The more hydrophilic fraction contained one major peak. The more hydrophobic fraction contained three peaks. The hydrophilic fraction accounted for about 75-85% of the AGP in all four peaks, and the hydrophobic fraction accounted for 15-25% (see FIG. 6). The two fractions were separated at about 15 minutes (see arrow on figure) or 20% acetonitrile. Other time points or acetonitrile concentrations that separate the peaks into the bimodal distribution are useful in the practice of this invention. In this example, the hydrophilic fraction was collected with the initial flow-through.

### Example 10

### Fostering Somatic Embryogenic Competence by AGP Hydrophilic and Hydrophobic Fractions

Coker 315 hypocotyls were grown on basic media (no hormones). After the callus was established, explants, were transferred to basic media with or without added hydrophilic AGP fraction (0.85 mg/L), or hydrophobic AGP fraction (0.15 mg/L) from Example 9. The concentrations of the fractions were selected to match their proportion in the total AGP, as determined in Example 9. In this experiment, the hydrophilic AGP fraction also contained the wash-through from the column which may have contained some of the hydrophobic fraction if the column was overloaded. The explants were transferred to fresh media every four weeks. At time intervals, the callus was scored for embryogenic callus formation.

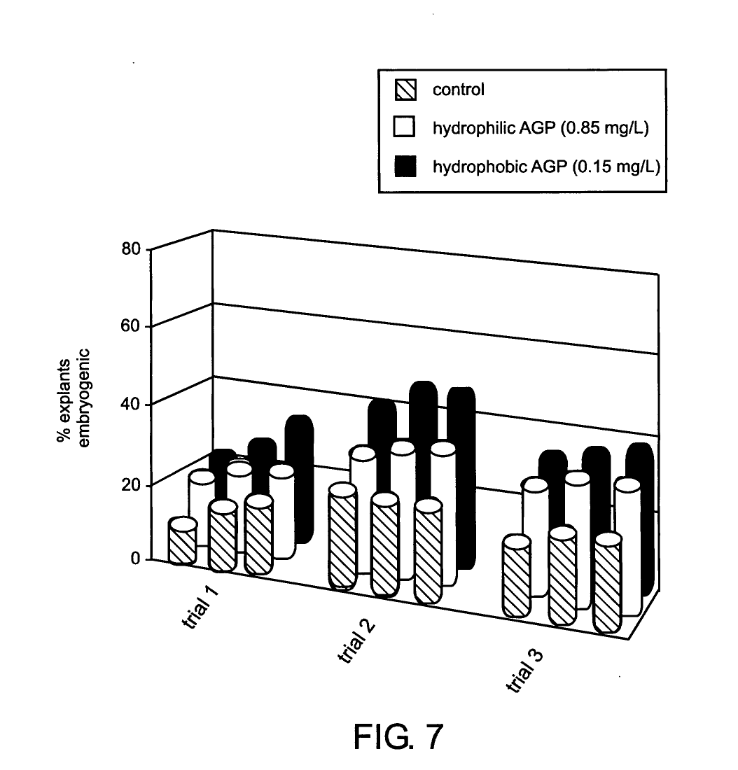

Table 6 and FIG. 7 show the results of three experiments of the percent of embryogenic explants. Between 75 and 102 explants were scored for each trial. Both the hydrophilic (with wash-through) and the hydrophobic fractions fostered somatic embryogenic competence, but the hydrophobic fraction was at least about 5× more active on a weight-for-weight basis.

TABLE 6
Hydrophilic and Hydrophobic Fractions of Embryogenic AGP

| | | | hydrophilic | hydrophobic |
|---|---|---|---|---|
| | | Control | AGP (0.85 mg/L) | AGP (0.\grave{ }15 mg/L) |
| Trial | Week | (%) | (%) | (%) |
| 1 | 4 | 8 | 18 | 18 |
| 1 | 6 | 15 | 22 | 25 |
| 1 | 8 | 18 | 23 | 33 |
| 2 | 4 | 24 | 31 | 40 |
| 2 | 6 | 24 | 34 | 46 |
| 2 | 8 | 24 | 35 | 46 |
| 3 | 4 | 19 | 29 | 33 |
| 3 | 6 | 22 | 32 | 35 |
| 3 | 8 | 22 | 32 | 37 |

### Example 11

### Fractionation of Embryogenic AGPs into Peaks

The total embryogenic AGP extracted in Example 2 was split into 4 peaks (labeled by time point arrows) by RP-HPLC as in Example 9, as is shown in FIG. 8. Fraction 1, containing hydrophilic peak #1, the first peak to elute, was 75% of the total amount of AGP in the four peaks. Three hydrophobic peaks, Fraction 2 containing hydrophobic peak #1, Fraction 3 containing hydrophobic peak #2, and Fraction 4 containing hydrophobic peak #3, represented 4%, 11% and 10%, respectively, of the total AGP by weight. Fraction 1 containing hydrophilic peak #1 eluted at 4-12% acetonitrile or 3-9 min. Fraction 2 containing hydrophobic peak #1 eluted at 27-32% acetonitrile or 20-23.5 min. Fraction 3 containing hydrophobic peak #2 eluted at 32-37% acetonitrile or 23.5 to 28 min. Fraction 4 containing hydrophobic peak #3 eluted at 44-49% acetonitrile or 33-37 min.

### Example 12

### Fostering Somatic Embryogenic Competence by AGP Hydrophilic and Hydrophobic Peaks

Coker 315 hypocotyls were grown on basic media (no hormones). After the callus was established, explants, were transferred to basic media with or without added Fraction 1, 2, 3 or 4 (Example 11). The concentrations of the AGP in the peaks were selected to match their proportion in the total AGP, as determined in Example 11. In this experiment, Fraction 1 did not contain the wash-through from the column. The explants were transferred to fresh media every four weeks. At time intervals, the callus was scored for embryogenic callus formation.

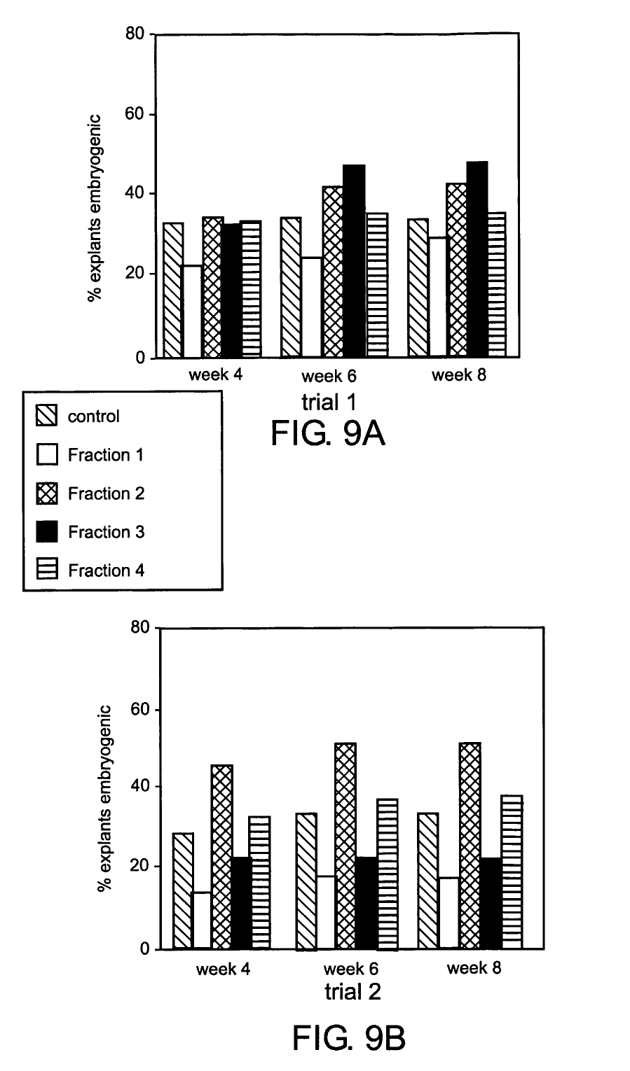

Table 7 and FIGS. 9A and 9B show the results of four experiments of the percent of embryogenic explants. Between 44 and 108 explants were scored for each trial. The concentrations of the peaks were selected to represent the same concentration of peak that was present in 2 mg/L total AGP. Fraction 1 had a slight inhibitory activity. Of the three hydrophobic peaks (Fractions 2-4), Fraction 2 had the highest competence fostering activity when averaged over all experiments. Fraction 4 had no activity. Fraction 3 had activity in two of the four experiments.

TABLE 7
RP-HPLC Peaks of Embryogenic AGP

| | | | Fraction 1 | Fraction 2 | Fraction 3 | Fraction 4 |
|---|---|---|---|---|---|---|
| | | Control | 1.5 mg/L | 0.08 mg/L | 0.22 mg/L | 0.2 mg/L |
| Trial | Week | (%) | (%) | (%) | (%) | (%) |
| 1 | 4 | 33 | 22 | 34 | 32 | 33 |
| 1 | 6 | 34 | 24 | 42 | 48 | 35 |
| 1 | 8 | 34 | 29 | 43 | 48 | 35 |
| 2 | 4 | 29 | 14 | 46 | 23 | 33 |
| 2 | 6 | 34 | 18 | 52 | 23 | 38 |
| 2 | 8 | 34 | 18 | 52 | 23 | 39 |
| 3 | 4 | 56 | 56 | 68 | 55 | 59 |
| 3 | 6 | 77 | 86 | 85 | 66 | 73 |
| 3 | 8 | 77 | 86 | 86 | 74 | 73 |
| 4 | 4 | 59 | 48 | 67 | 68 | 64 |
| 4 | 6 | 69 | 62 | 81 | 77 | 72 |
| 4 | 8 | 87 | 80 | 93 | 89 | 79 |

### Example 13

### Carbohydrate Characterization of Non-embryogenic and Embryogenic Total AGP and De-arabinosylation and De-glycosylation of Total AGP

Carbohydrate accounts for the major component of most AGPs. The monosaccharide composition of both the non-embryogenic and the embryogenic AGPs was analyzed. The monosaccharide compositions were determined using alditol acetates (Albersheim et al. 1967 Carbohydrate Res. 5, 340-345; Blakeney et al. 1983 Carbohydrate Res. 113, 291-299). Both had Ara and Gal as the major monosaccharides in the ratio of 2:1 (see Table 8), which is typical of classical AGPs.

Non-embryogenic and embryogenic total AGPs (example 2) were deglycosylated using anhydrous hydrofluoric acid (HF) according to the method described in Mau et al. (1995) Plant J. 8, 269-281. AGPs (17.6 mg) were de-arabinosylated by incubating in 0.2 M TFA (8.8 mL) at 100° C. for 2 hours. The mixture was then cooled and the TFA removed by rotary evaporation. The sample was then applied to a pre-packaged Sephadex G-25 M PD-10 column (Amersham Biosciences, Piscataway, N.J., USA). Purified TFA-treated AGP was eluted with water and lyophilized. 5.4 mg TFA-treated AGP was obtained. Mild hydrolysis with TFA removed Ara preferentially. The de-arabinosylated AGP was isolated and analyzed and, as expected, only Gal was detected (see Table 8).

TABLE 8
Carbohydrate Characterization of AGP

| | Non-emb | Emb | TFA treated |
|---|---|---|---|
| | AGP | AGP | AGP |
| sugar | (%) | (%) | (%) |
| Gal | 65 | 64 | 99 |
| Ara | 33 | 30 | trace |
| Rha | 1 | 3 | — |
| Glu | 1 | 1 | 1 |
| Man | — | 1 | trace |
| Xyl | — | 1 | trace |

Both non-embryogenic and embryogenic total AGP preparations stained with Yariv reagent on a gel and both were very high molecular weight, typical of AGPs. De-glycosylation by TFA removed some of the carbohydrate reducing the Yariv-binding properties while de-glycosylation by HF removed all the carbohydrate and the remaining protein backbone behaved on a gel as though it had a molecular weight of about 26 kD.

### Example 14

### Fostering Somatic Embryogenic Competence by Deglycosylated and De-arabinosylated AGP

Coker 315 hypocotyls were grown on basic media (no hormones). After the callus was established, explants were transferred to basic media with or without added deglycosylated or de-arabinosylated AGP (Example 13). The concentrations of the deglycosylated or de-arabinosylated AGP were difficult to quantitate directly, and were therefore expressed as concentration of the respective AGP prior to treatment: 1 mg/L in trial 1 and of 2 mg/L in trial 2. The explants were transferred to fresh media every four weeks. At time intervals, the callus was scored for embryogenic callus formation.

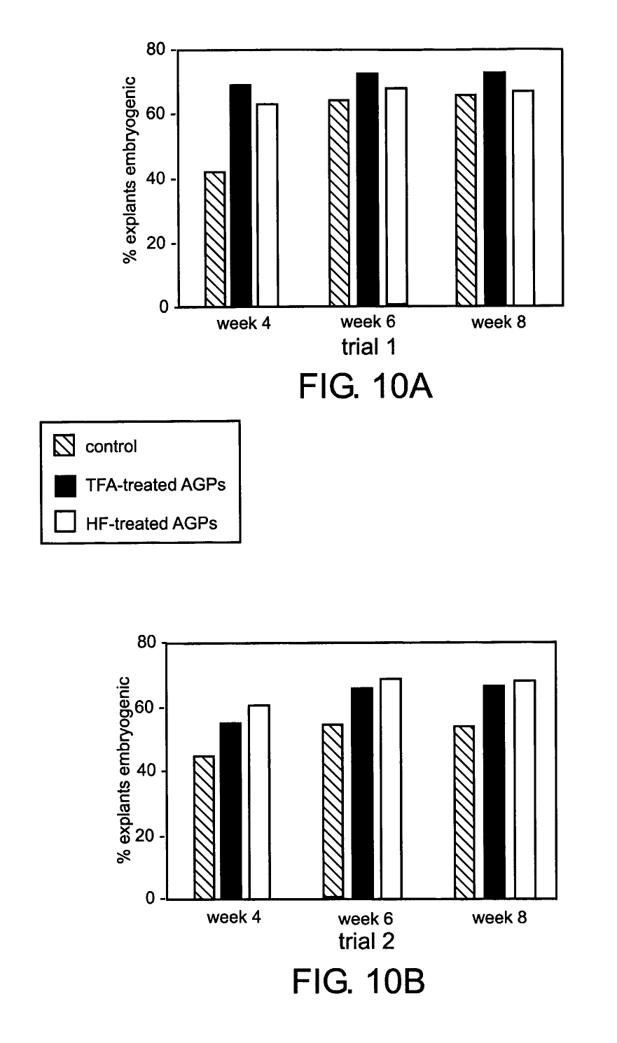

Table 9 and FIGS. 10A and 10B show the results of the percent of embryogenic explants. Between 66 and 105 explants were scored for each trial. Both deglycosylated and de-arabinosylated AGP fostered somatic embryogenic competence.

TABLE 9
Deglycosylated and De-arabinosylated AGP

| | | Control | De-arabinosylated | Deglycosylated |
|---|---|---|---|---|
| Trial | Week | (%) | AGP (%) | AGP (%) |
| 1 | 4 | 43 | 70 | 64 |
| 1 | 6 | 65 | 73 | 68 |
| 1 | 8 | 67 | 73 | 68 |
| 2 | 4 | 46 | 55 | 61 |
| 2 | 6 | 54 | 66 | 68 |
| 2 | 8 | 54 | 67 | 68 |

### Example 15

### Fostering Somatic Embryogenic Competence Using Commercial Cotton Varieties

Embryogenic total AGP (Example 2) was assayed for fostering somatic embryogenic competence using hypocotyls from four commercial cultivars, Emerald, Siokra 1-4, Sicala 40, and Sicot 189. Five different hormone combinations (A-E) were tested in the media.

All hormone-containing media were based on the basic media containing MS salts, Gamborg's vitamins, glucose, potassium nitrate, magnesium chloride hexahydrate, myo-inositol, gellan gum, pH 5.8.

Medium A

0.1 mg/L kinetin (KT)
0.1 mg/L 2,4-dichlorophenoxyacetic acid (2,4-D)

Medium B

2 mg/L a-naphthaleneacetic acid (NAA)
0.044 mg/L 2,4-D
(See “MCIM” in Mishra R, Wang H Y, Yadav N R, Wilkins T A. (2003) Development of a highly regenerable elite Acala cotton (Gossypium hirsutum cv. Maxxa)—a step towards genotype-independent regeneration. Plant Cell Tiss. Org. Culture 73:21-35.)

Medium C

2 mg/L NAA
0.044 mg/L 2,4-D
0.086 mg/L KT

### (See “MCIM+K” in Mishra et al. (2003).)

Medium D

0.5 mg/L KT
1 mg/L indole-3-butyric acid (IBA)
Poster abstract by Wu J, et al. 3rd World Cotton Research Conference, Cape Town, South Africa, 9-13 Mar. 2003 described a medium comprising 1 mg/L IBA and 0.5 mg/L KT, however, there were incomplete details of the other medium components. Wu et al. also described transferring the callus produced on medium containing the above hormones to a medium with lower concentrations of hormones (0.5 mg/L IBA and 0.3 mg/L KT) as well as increases in MgSO₄ and FeSO₄ to promote the conversion to embryogenic callus, but this is not necessary in the practice of this invention.

Medium E

0.5 mg/L KT
2 mg/L NAA

### (See Sakhanokho et al. (2001), which uses 1 mg/L KT instead of the 0.5 mg/L, but the same amount of NM).

Hypocotyls were grown on basic media without added hormones and with hormone cocktail A, B, C, D, or E. After the callus was established, explants, were transferred to fresh media with or without added AGPs extracted from embryogenic callus, Example 2. The concentration of AGP was about 2 mg/L. The explants were transferred to fresh media every four weeks. At time intervals, the explants were scored for callus formation and for embryogenic callus formation. Embryogenic callus was not obtained from Emerald, Sicala 40 or Sicot 189, whether AGP was present or not.

### Example 16

### Fostering Somatic Embryogenic Competence in Siokra 1-4 Using AGP Without Hormones

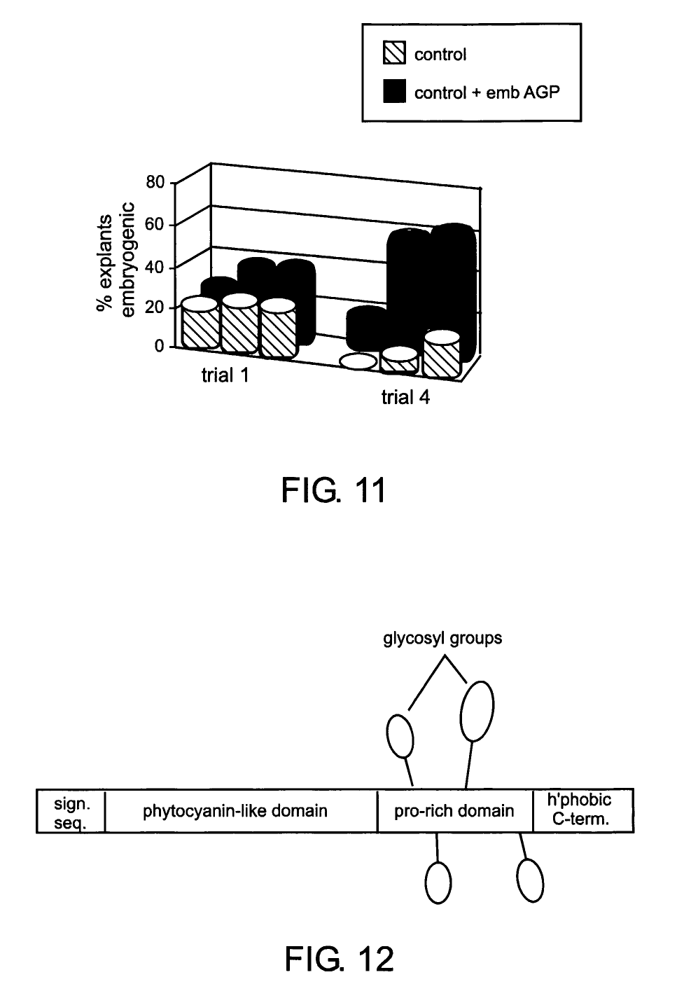

Table 10 and FIG. 11 show the percentage of embryogenic explants at various time points for two experiments. Siokra 1-4 cotton is sold commercially for dryland planting. Siokra 1-4 hypocotyls were grown on basic media without added hormones and with or without about 2 mg/L embryogenic AGP (Example 2). After the callus was established, explants were transferred to fresh media with or without AGPs at about 2 mg/L extracted from Coker 315 embryogenic callus, Example 2. The explants were transferred to fresh media, every four weeks. At time intervals, between 18 and 105 explants were scored for embryogenic callus formation. When hypocotyls were initially on basic media without AGP, contacting with AGP fostered somatic embryogenic competence, similar to the effect using Coker 315 explants. However, when AGP was added to the basic media initially, although somatic embryogenic competence appeared to be impeded in the control (no contact with AGP after initial contact), somatic embryogenic competence was fostered even more when continuing contact with AGP. Consequently, although contacting hypocotyls with AGP initially may appear to impede somatic embryogenic competence, continued contacting with AGP after initially contacting substantially fostered somatic embryogenic competence, more than any other combination tested in this experiment.

TABLE 10
AGP Fostering Somatic Embryogenic Competence in Siokra 1-4

| | Hypocotyls Initially | | Control | AGP |
|---|---|---|---|---|
| Trial | on AGP | Week | (%) | (%) |
| 1 | No | 4 | 20 | 24 |
| | No | 6 | 24 | 37 |
| | No | 8 | 24 | 37 |
| 2 | No | 4 | 16 | 14 |
| | No | 6 | 22 | 28 |
| | No | 8 | 25 | 28 |
| 3 | No | 4 | 12 | 18 |
| | No | 6 | 17 | 26 |
| | No | 8 | 23 | 34 |
| 4 | Yes | 4 | 0 | 17 |
| | Yes | 6 | 6 | 56 |
| | Yes | 8 | 17 | 61 |

### Example 17

### Fostering Somatic Embryogenic Competence in Siokra 1-4 Using Medium D

Siokra 1-4 hypocotyls were grown on basic media with hormone cocktail D. After five weeks, explants, were transferred to fresh media with or without added AGPs at about 2 mg/L extracted from embryogenic callus, Example 2. The explants were transferred to fresh media, every four weeks. At time intervals, the explants were scored for embryogenic callus formation. About 45 explants were tested. Somatic embryogenic callus was produced after 8 weeks both with and without added AGP (see Table 11), but was produced to a greater extent with AGP.

TABLE 11
Fostering Somatic Embryogenic Competence in
Siokra 1-4 Using Hormone IBA and KT

| Weeks | Control Cocktail D | AGP added |
|---|---|---|
| 8 | 21% | 24% |
| 12 | 24% | 31% |

### Example 18

### Fostering Somatic Embryogenic Competence in Sicala 40 Using AGP Without Hormones

Sicala 40 hypocotyls were grown on basic media without added hormones. After four weeks, 44 to 45 explants were transferred to fresh media with or without added AGPs at about 2 mg/L from embryogenic callus, Example 2. After four, six and eight weeks, the explants were scored for embryogenic callus formation (see Table 12). Without AGP, none of the Sicala 40 hypocotyl segments produced any callus after four weeks, and none of the original callus was healthy 4 weeks after the first transfer from callus induction media. With added embryogenic AGP, about 16% of the Sicala 40 explants produced embryogenic callus at eight weeks.

TABLE 12
AGP Fostering Somatic Embryogenic Competence in Sicala 40

| | Control Cocktail D | AGP |
|---|---|---|
| 4 weeks | 0% | 11% |
| 6 weeks | 0% | 16% |
| 8 weeks | 0% | 16% |

### Example 19

### Regeneration of Siokra 1-4 and Sicala 40

Siokra 1-4 somatic embryos fostered using AGP in Example 17 and Sicala 40 somatic embryos fostered using AGP in Example 18 are regenerated into fertile plants, allowed to self pollinate, and viable seed is harvested.

### Example 20

### Fostering Somatic Embryogenic Competence by AGP Using Various Plant Tissues and Cell Types

Coker 315 petioles and leaves were assayed with or without hormone cocktail A, B, C, D, or E, and then were transferred onto hormone free media with or without AGPs.

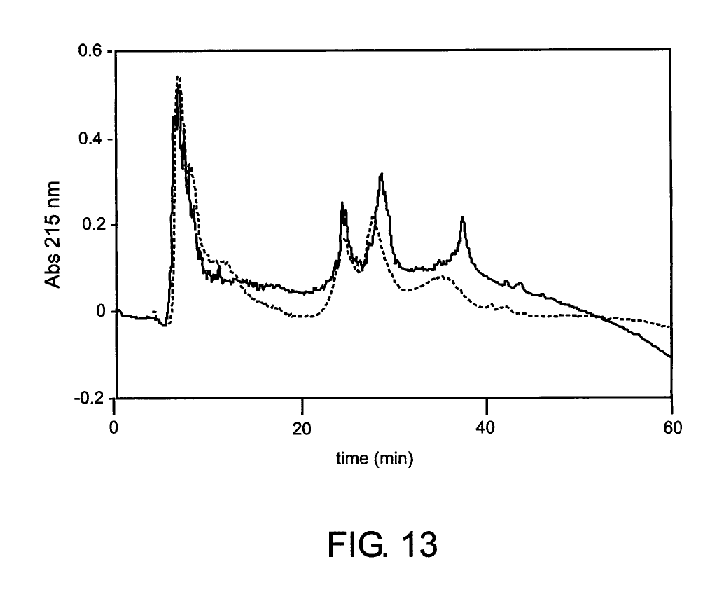

Coker 315 petioles were grown on basic media without added hormones and with hormone cocktail A, B, C, D, or E (FIGS. 13A-13J). After five weeks, explants were transferred to fresh media with or without added AGPs extracted from embryogenic callus, Example 2. The concentration of AGP was about 2 mg/L. The explants were transferred to fresh media, with or without AGP, every four weeks. At time intervals, the explants were scored for callus formation and for embryogenic callus formation. Callus survived for about four weeks longer in the presence of AGPs, but everything eventually died, except with hormone cocktail D, which became embryogenic regardless of the presence or absence of AGPs see FIGS. 13G and 13H).

Coker 315 leaves were grown on basic media without added hormones and with hormone cocktail A, B, C, D, or E, and with or without AGP extracted from embryogenic callus, Example 2. After five weeks, explants were transferred to hormone free media with or without added AGPs extracted from embryogenic callus, Example 2. The concentration of AGP was about 2 mg/L. The explants were transferred to fresh media, every four weeks. At time intervals, the explants were scored for callus formation and for embryogenic callus formation. Callus was produced on several combinations of hormones as well as on hormone free media. Inclusion of the AGP in the media resulted in about 25% more rapid formation of embryogenic callus (FIGS. 14A-14C), or formation of an equivalent percentage of embryogenic callus in about six weeks instead of about eight weeks, depending on the hormone cocktail. FIG. 14A shows embryogenic callus produced after contacting with AGP containing media after transfer from callus induction media with AGP. FIG. 14B shows callus produced using hormone cocktail D followed by contacting with AGP. FIG. 14C shows embryogenic callus produced without AGP after callus induction using hormone cocktail B. Contacting leaves with AGP fostered somatic embryogenesis.

### Example 21

### Characterization of Total AGP from Embryogenic Callus Cultured with Agp

After 5 weeks on medium #1 and then 8 weeks on medium #1 with 1 mg/L total AGP, embryogenic callus tissue and embryos were harvested. The embryos were regenerated. AGPs were extracted from the embryogenic callus tissue according to the method used in Example 2. Total AGPs were fractionated by RP-HPLC as in Example 3. The RP-HPLC profile was similar, including peak distribution and size, compared to that of AGPs from embryogenic callus grown on media without added AGP.

### Example 22

### Fostering Somatic Embryogenic Competence in Other Cotton Species

Explants from Pima cotton, Sea Island cotton, and Egyptian cotton varieties that are recalcitrant to regeneration are contacted with Coker 315 embryogenic callus hydrophobic peak #1 AGP at a concentration of 0.08 mg/L resulting in fostering somatic embryogenic competence. An explant from a wild relative of cultivated cotton, Gossypium thurberi, is contacted with Coker 315 embryogenic callus total AGP at a concentration of 1.5 mg/L resulting in fostering of somatic embryogenic competence. Indigenous Australian cotton species G. sturtianum, G. robinsonii, Gossypium australe, and Gossypium bickii are contacted with Coker 315 embryogenic callus hydrophobic peak #1 AGP at a concentration of 0.08 mg/L resulting in fostering somatic embryogenic competence. Explants from tree cotton, Creole cotton, Levant cotton, Sturt's desert rose, Thurber's cotton, and Hawaii cotton are contacted with Coker 315 embryogenic callus hydrophobic peak #1 AGP at a concentration of 0.08 mg/L resulting in fostering somatic embryogenic competence.

### Example 23

### Fostering Somatic Embryogenic Competence in Malvales

Explants from Okra and Hibiscus are contacted with Coker 315 embryogenic callus hydrophobic peak #1 AGP at a concentration of 0.08 mg/L resulting in the fostering somatic embryogenic competence.

### Example 24

### Isolation of Peptides from AGP Hydrophobic Peaks

It is known that AGP fractions can contain AGPs and other proteins that co-purify together with the AGPs, using Yariv reagent extraction and RP-HPLC. Related AGPs can co-elute from an RP-HPLC column. The AGP RP-HPLC peaks are somewhat broad, and broad peaks can comprise several proteins or several forms of a protein.

Tryptic digestion was performed on Fraction 2 containing hydrophobic peak #1 (Example 11), without de-glycosylation or de-arabinosylation. For tryptic digestion, RP-HPLC-purified and lyophilised AGP (<1 mg) was solubilized in 50 mM ammonium bicarbonate (467 μL) (pH 7.8). A 20 μg aliquot of Sequencing Grade Modified Trypsin (Promega, catalogue no. V5111) was solubilized in 50 mM acetic acid (100 μL) and heated at 30° C. for 15 min. An aliquot of the trypsin solution (33 μL) was then added to the AGP solution and the mixture was incubated at 37° C. for 16 h. The peptides from the digested AGPs were then purified by RP-HPLC, as described herein, dried and N-terminally sequenced by Edman degradation (LF3000 Series Protein Sequencer, Beckman). Three peptide sequences were obtained, and are listed in Table 13.

TABLE 13

| SEQ ID NO | Sequence |
|---|---|
| 1 | EDYSXXTSNPIAEYK |
| 2 | IQIGDSLV |
| 3 | STASLGVTLSV |

Tryptic digestion was performed on Fraction 3 containing hydrophobic peak #2 (Example 11), without de-glycosylation or de-arabinosylation. The peptide products of the trypsin digest were sequenced. Four peptide sequences were obtained, and are listed in Table 14.

TABLE 14

| SEQ ID NO | Sequence |
|---|---|
| 4 | AGTLRPEKPFTAN |
| 5 | DGWVVSPSENYNHWAE |
| 6 | IQVXDEVXE |
| 7 | YAGDTITGNTDNS |

### Example 25

### Cloning of Genes Encoding Tryptic Peptides

Genes encoding proteins comprising peptides having sequences of SEQ ID NOS:1-2, 4 and 5 were cloned.

RNA was isolated from embryogenic cofton callus using Trizol LS reagent (Gibco BRL, catalogue no. 10296-010).

The RNA was used to synthesize cDNA using an oligo-dT primer from a 3′RACE System for Rapid Amplification of cDNA Ends Kit (Invitrogen Life Technologies, Carlsbad, Calif., USA, catalog no. 18373-019) and SuperScript II Reverse Transcriptase (Invitrogen Life Technologies, Carlsbad, Calif., USA, catalogue no. 18064-022).

Degenerate oligonucleotide primers based on the peptide sequences of trypsin-digested AGPs were designed. A combination of the following sequences yielded products (I=inositol):

| | |
|---|---|
| primer (SEQ ID NO:8) | 5′ AAC/T CCI ATI GCI GAG/A TAT/C AA 3′ |
| was designed to anneal to DNA encoding: | N P I A E Y K |
| which was present in SEQ ID NO:1, | |
| and | |
| primer (SEQ ID NO:9) | 5′ AAC/T TAC/T AAC/T CAT TGG GCI GA 3′ |
| was designed to anneal to DNA encoding: | N Y N H W A E |
| which was present in SEQ ID NO:5. | |
| Primer (SEQ No:10) | 5′ CCI CAG/A AAG/A CCI TTT/C ACI GCI AA 3′ |
| was designed to anneal to DNA encoding: | P E K P F T A N |
| which was present in SEQ ID NO:4 | |

3′ Rapid Amplification of cDNA Ends (3′RACE) was performed using one of the above primers in conjunction with a reverse primer based on the sequence of the oligo-dT primer. cDNA synthesized in step 2 was used as the template for PCR while the enzyme was Taq DNA Polymerase (Invitrogen Life Technologies, Carlsbad, Calif., USA, catalogue no. 18038-042).

Resultant DNA fragments were gel purified (QIAEX II Gel Extraction Kit, catalogue number 20021) and cloned into the vector, pGEM-T EASY (Promega, Madison, Wis., USA, catalogue no. A1360). DNA from resultant clones was sequenced (Australian Genome Research Facility, Brisbane).

A DNA fragment encoding a peptide comprising the amino acid sequences of SEQ ID No:4 was obtained. The nucleotide sequence of the fragment is shown as SEQ ID No: 11.

### SEQ ID NO:11 (GhPRP1 partial nucleotide sequence (84 bases))

1 CCCCAGAAGC CATTTACTGC GAACAAGCTT CCGTTTATTC TCTACACCGT
51 TGGGCCATTT GCTTTCGAAC CCAAATGCTA CTAG

The encoded amino acid sequence of 27 amino acids is given in SEQ ID No:12

1 PEKPFTANKL PFILYTVGPF AFEPKCY-

[DNA and amino acid sequence fragments of SEQ ID No:11 and SEQ ID NO:12 were designated GhPRP1 which refers to proline-rich protein which is what this sequence is similar to in the data base. Attempts to clone a full length gene by 5′RACE have been unsuccessful to date.]

Nested oligonucleotide primers based on the partially cloned sequences were then designed. For GhCAGP1 (previously named GhEmbAGP1), the outer primer was: 5′GCT ATT TCT ATA GCA ACT CAA C 3′ (SEQ ID NO:13), and the inner primer was: 5′CAA ACT CAA AAC AAC CCC AAA ACC 3′ (SEQ ID NO: 14). For GhCAGP2 (previously named GhEmbAGP2), the outer primer was: 5′GAT GAA AGC AAG GCA CAC ACA C 3′ (SEQ ID NO:15), and the inner primer was: 5′CCC CTT AAT AAT TCA GCA CC 3′ (SEQ ID NO:16). These primers were used in PCR reactions to amplify from the 3′ end to the 5′ end of the genes in conjunction with the appropriate nested primers provided in the FirstChoice RLM-RACE kit (Ambion, Austin, Tex., USA, catalogue no. 1700) using 5′ RNA Ligase Mediated Rapid Amplification of cDNA Ends (5′ RLM-RACE). The reaction was performed using the FirstChoice RLM-RACE kit (Ambion, Austin, Tex., USA, catalogue no. 1700) based on the manufacturer's instructions. Gel purification, cloning into pGEM-T EASY and DNA sequencing was performed.

PCR protocols, including RACE, are known in the art (PCR protocols, edited by John M. S. Bartlett and David Stirling, 2nd edition, Totowa, N.J., Humana Press, 2003; and PCR cloning protocols, edited by Bing-Yuan Chen and Harry W. Janes 2nd edition, Publisher Totowa, N.J.: Humana Press, 2002).

The sequence of the protein comprising peptides having sequences in SEQ ID NOS:1-2 is listed in SEQ ID NO:18, and the gene has been named GhCAGP1, for Gossypium hirsutum chimeric AGP #1. The sequence of the protein comprising a peptide having the sequence in SEQ ID NO:5 is listed in SEQ ID NO:20, and the gene has been named GhCAGP2, for Gossypium hirsutum chimeric AGP #2. Both have four domains, as shown in FIG. 12 and as listed below: a signal sequence, a phytocyanin-like domain, a pro-rich domain, and a hydrophobic C-terminal tail. The gene sequences encoding SEQ ID NOS:18 and 20 are listed in SEQ ID NOS:17 and 19, respectively. SEQ ID NO:1 corresponds to amino acid numbers 79-94 of SEQ ID NO:18; SEQ ID NO:2 corresponds to amino acid numbers 56-63 of SEQ ID NO:18; and SEQ ID NO:5 corresponds to amino acid numbers 33-38 of SEQ ID NO:20.

```
SEQ ID NO:17 (GhCAGP1 nucleotide sequence (528 bases))
    1 ATGGCTGCTA AAGCTTTTTC AAGAAGTATA ACTCCTTTGG TGCTTTTGTT
  51 CATATTTTTA AGCTTTGCAC AAGGTAAAGA AATCATGGTT GGTGGCAAAA
101 CAGGCGCTTG GAAGATACCT TCTTCTGAAT CAGATTCTCT CAACAAATGG
151 GCTGAAAAAG CTCGTTTCCA AATCGGCGAC TCTCTCGTGT GGAAATATGA
201 TGGTGGTAAA GACTCGGTGC TCCAAGTGAG TAAGGAGGAT TATACAAGTT
251 GCAATACGTC GAACCCGATT GCCGACTACA AAGATGGGAA CACCAAGGTG
301 AAGCTTGAAA AGTCAGGACC ATATTTCTTC ATGAGTGGAG CAAAGGGCCA
351 CTGCGAGCAA GGCCAGAAGA TGATTGTGGT TGTGATGTCT CAAAAGCATA
401 GGTACATTGG AATCTCTCCA GCACCTTCGC CGGTTGATTT TGAAGGTCCG
451 GCCGTTGCTC CAACAAGCGG AGTTGCAGGG TTGAAGGCTG GTTTGTTGGT
501 GACAGTGGGG GTTTTGGGGT TGTTTTGA
SEQ ID NO:18 GhCAGP1 amino acid sequence (175 AA)
    1 MAAKAFSRSI TPLVLLFIFL SFAQGKEIMV GGKTGAWKIP SSESDSLNKW
  51 AEKARFQIGD SLVWKYDGGK DSVLQVSKED YTSCNTSNPI AEYKDGNTKV
101 KLEKSGPYFF MSGAKGHCEQ GQKMIVVVMS QKHRYIGISP APSPVDFEGP
151 AVAPTSGVAG LKAGLLVTVG VLGLF-
```

The signal sequence is located at amino acids 1-25 (nucleotide bases 1-75). The phytocyanin-like domain is located at amino acids 26-138 (nucleotide bases 76-414). The pro-rich domain is located at amino acids 139-156 (nucleotide bases 415-468). The hydrophobic C-terminal tail is located at amino acids 157-175 (nucleotide bases 469-525). The peptides corresponding to and having sequences similar to SEQ ID NOS:1, 2 and 5 are shown in bold.

```
SEQ ID NO:19 (GhCAGP2 nucleotide sequence (660 bases))
    1 ATGGGGTTCG AAAGGTATCT TGCTAGTGTG TTGATAGTGA TAATGCTGTC
  51 TTTTATCACT TCATCACAGG GTTATAAGTT CTATGTTGGT GGTAGAGACG
101 GTTGGGTTGT TAGTCCTTCT GAGAACTACA ATCATTGGGC TGAAAGGAAT
151 AGATTCCAAG TCAATGATAC TCTCTTTTTC AAGTACAAGA AAGGGTCAGA
201 CTCGGTGCTG TTGGTAACAA GAGAAGATTA CTTCTCATGC AACACCAAGA
251 ACCCAATTCA GTCTTTAACA GAAGGTGATT CACTCTTTAC ATTTGATCGG
301 TCGGGTCCCT TCTTTTTCAT CACCGGTAAC GCTGATAATT GCAAAAAAGG
351 GCAAAAGCTG ATCGTCGTGG TCATGGCTGT AAGACACAAA CCCCAGCAAC
401 AACCTCCTTC ACCTTCTCCC TCATCTGCTG TGACAACAGC GCCGGTTTCT
451 CCACCCACAT TACCCATTCC TGAAACTAAC CCTCCTGTAG AGTCACCAAA
501 GAGCAGTGAG GCTCCATCTC ATGATGCTGT GGAACCAGCT CCGCCGGAGC
551 ACAGATCGGG TTCATTCAAA CTAGTATGTT CTACCTGGCT GGTGTTGGGT
601 TTCGGCATTT GGGTCAGCAT GGCCTTGGGG ATCGAAAATG TAGTTTGTTT
651 TTGGTGCTGA
SEQ ID NO:20 GhCAGP2 amino acid (219 AA)
    1 MGFERYLASV LIVIMLSFIT SSQGYKFYVG GRDGWVVSPS ENYNHWAERN
  51 RFQVNDTLFF KYKKGSDSVL LVTREDYFSC NTKNPIQSLT EGDSLFTFDR
101 SGPFFFITGN ADNCKKGQKL IVVVMAVRHK PQQQPPSPSP SSAVTTAPVS
151 PPTLPIPETN PPVESPKSSE APSHDAVEPA PPEHRSGSFK LVCSTWLVLG
201 FGIWVSMALG IENVVCFWC-
```

The signal sequence is located at amino acids 1-24 (nucleotide bases 1-72). The phytocyanin-like domain is located at amino acids 25-130 (nucleotide bases 73-390). The pro-rich domain is located at amino acids 131-182 (nucleotide bases 391-546). The hydrophobic C-terminal tail is located at amino acids 183-219 (nucleotide bases 547-657). The peptide corresponding to and having a sequence similar to SEQ ID NO:5 is shown in bold.

### Example 26

### Protein Sequencing Without Tryptic Digestion

The protein backbones of AGPs in Fraction 2 were sequenced without digesting the proteins with trypsin. Fraction 2 yielded the peptide sequence KEIMVGGKTGAWKIP (SEQ ID NO: 27), which matched the predicted N-terminal sequence of the mature protein (i.e., without the signal sequence), amino acids 26-40 of SEQ ID NO:18.

### Example 27

In Example 14, deglycosylated and de-arabinosylated embryogenic AGP were shown to be active in fostering embryogenesis. The cloned embryogenic AGP genes GhCAGP1 and 2 both have phytocyamin-like (PL) domains, as noted in Example 25. The respective PL domains were amplified for expression in bacteria.

The primers:

(SEQ ID NO:21)

5′ CACCCTGGTTCCGCGTGGATCCAAAGAAATCATGGTTGGTGGCAAAA
C 3′
and

(SEQ ID NO:22)

5′ CTAGATTCCAATGTACCTATGCTTTTGAGAC 3′

were used to amplify the PL domain from GhCAGP1.
The primers:

(SEQ ID NO:23)

5′ CACCCTGGTTCCGCGTGGATCCTATAAGTTCTATGTTGGTGGTA
G 3′
and

(SEQ ID NO:24)

5′ CTATTGTTGCTGGGGTTTGTGTCTTACAGCCATG 3′

were used to amplify the PL domain from GhCAGP2.

DNA encoding thrombin cleavage sites were incorporated at the 3′ ends of the forward primers. The enzymes used to amplify the DNA were either Platinum Pfx DNA polymerase or Platinum Taq High Fidelity (Invitrogen Life Technologies, Carlsbad, Calif., USA, catalogue numbers 11708-013 and 11304-011). PCR products were cloned using the pENTR/D-TOPO Cloning Kit (Invitrogen Life Technologies, Carlsbad, Calif., USA, catalogue number 45-0218) and then transferred into the expression vector, pDEST17, for expression of the proteins with an N-terminal histidine tag (Invitrogen Life Technologies, Carlsbad, Calif., USA, catalogue number 11803-012). This was then used to transform BL21 Star (DE3) One Shot Escherichia coli cells (Invitrogen Life Technologies, Carlsbad, Calif., USA, catalogue number C6010-03). Expressed proteins were purified using Ni-NTA Agarose (QIAGEN GmbH, Hilden, Germany, catalogue number 30210); yields of the purified, recombinant proteins PL1 and PL2 were 35 mg/L bacterial cell culture and 25 mg/L bacterial cell culture, respectively.

The sequences of recombinant PL1 and PL2 were:

PL1: (SEQ ID NO:25)
MSYYHHHHHH LESTSLYKKA GSAAAPFTLV PRGSKEIMVG
GKTGAWKIPS SESDSLNKWA EKARFQIGDS LVWKYDGGKD
SVLQVSKEDY TSCNTSNPIA EYKDGNTKVK LEKSGPYFFM
SGAKGHCEQG RKMIVVVMSQ KHRYIGI
PL2: (SEQ ID NO:26)
MSYYHHHHHH LESTSLYKKA GSAAAPFTLV PRGSYKFYVG
GRDGWVVSPS ENYNHWAERN RFQVNDTLFF KYKKGSDSVL
LVTREDYFSC NTKNPIQSLT EGDSLFTFDR SGPFFFITGN
ADNCKKGQKL IVVVMAVRHK PQQQ

The N-terminal tags were removed using the Thrombin CleanCleave Kit (Sigma, St Louis, Mo., USA, catalogue number RECOM-T); cleavage was at R32-G33 of the recombinant proteins. Cleaved proteins were analysed by reversed-phase HPLC, mass spectrometry and N-terminal protein sequencing and then tested in the embryogenesis bioassay at a concentration of 0.5 mg/L.

### Example 28

The expressed PL-1 and PL-2 proteins were tested for activity to foster somatic embryogenic competence. Embryogenesis was tested as described in Example 4, using 0.5 mg/L protein. The results are given in Table 15.

TABLE 15
Embryogenesis Fostered by Bacterially Expressed Proteins
% Explants having embryogenic callus

| | | Control | Phytocyanin 1 | Phytocyanin 2 |
|---|---|---|---|---|
| Trial 1 | Week 4 | 18 | 41 | 21 |
| | Week 6 | 32 | 50 | 35 |
| | Week 8 | 41 | 62 | 52 |
| Trial 2 | Week 4 | 47 | 60 | 69 |
| | Week 6 | 51 | 65 | 80 |
| | Week 8 | 56 | 66 | 90 |

The results demonstrate embryogenesis-fostering activity for both thrombin-pretreated PL-1 and PL-2 at the concentration of 0.5 mg/L. Removal of the N-terminal tags is optional. Embryogenesis-fostering activity is observed in both PL-1 and PL-2 without thrombin pre-treatment. Embryogenesis can be maximally fostered by use of higher protein concentration, by combining PL-domain proteins, by use of PL-domain proteins of other AGP sources, and by other such expedients known to those skilled in the art, and as taught herein.

### Example 29

### Extraction of Embryogenic AGP from Siokra 1-4

AGPs were extracted from embryogenic Siokra 1-4 callus (method of Example 2). The HPLC profile was compared to pro-embryogenic Coker AGPs (FIG. 17). The profiles were similar, except that Hydrophobic Peak #3 had a slightly different retention time and shape, but this peak is slightly variable in extractions from Coker.

### Example 30

### Fostering Embryogenic Competence Using Siokra 1-4 AGPs

Coker 315, Siokra 1-4 and Sicala 40 hypocotyl explants are grown on basic media without added hormones and with or without about 2 mg/L pro-embryogenic AGPs from Siokra 1-4 (Example 29).

It will be appreciated by those of ordinary skill in the art that plant tissue culture methods and conditions, growing embryogenic callus, inducing callus, tissue culture media, hormone cocktails, AGP extraction methods, fractionation methodologies, AGP quantitation methods, RP-HPLC methods and materials, elution times and/or acetonitrile concentrations for dividing AGP into hydrophilic and hydrophobic fractions, resting seeds, AGP concentrations, species, varieties, cells, tissues, AGP source tissues, de-arabinosylation and de-glycosylation methods, regeneration methods, and transformation methods other than those specifically disclosed herein are available in the art and can be employed in the practice of this invention. All art-recognized functional equivalents are intended to be encompassed within the scope of this invention.

1. A method for fostering somatic embryogenic competence of a plant cell or tissue comprising contacting said plant cell or tissue with a pro-embryogenic arabinosylated protein (AGP) composition comprising a hydrophobic fraction of embryogenic AGP and maintaining the cell or tissue in culture to allow the cell or tissue to undergo somatic embryogenesis.

2. The method of claim 1 wherein the plant cell or tissue is of cotton.

3. The method of claim 1 wherein the plant cell or tissue is selected from the group consisting of Upland cotton, Pima cotton, Egyptian cotton, Sea Island cotton, G. hirsutum, G. barbadense, tree cotton, Creole cotton, Levant cotton, Sturt's desert rose cotton, Thurber's cotton, and Hawaii cotton.

4. The method of claim 1 wherein the plant cell or tissue is of an elite cotton line.

5. The method of claim 1 wherein the pro-embryogenic AGP composition comprises embryogenic AGP of a cotton variety.

6. The method of claim 1 wherein said AGP composition comprises AGP hydrophobic peak #1 from embryogenic callus from a cotton variety selected from the group consisting of Coker 315, Siokra 1-4, and Sicala 40 at a concentration between about 0.008 and about 0.8 mg/L, and wherein said plant cell or tissue is of a cotton variety that is recalcitrant to somatic embryogenesis.

7. The method of claim 5 wherein the pro-embryogenic AGP composition comprises embryogenic AGP selected from the group consisting of de-glycosylated AGP and de-arabinosylated AGP.

8. The method of claim 5 wherein the pro-embryogenic AGP is a protein having the amino acid sequence of SEQ ID NO: 25 or SEQ ID NO: 26.

9. The method of claim 5 wherein the pro-embryogenic AGP is a thrombin digest of the protein of SEQ ID No: 25.

10. The method of claim 5 wherein the pro-embryogenic AGP is a thrombin digest of the protein of SEQ ID No: 26.

11. A method for regenerating a plant comprising:
a) harvesting a plant cell or tissue from a first plant;
b) contacting said plant cell or tissue with an AGP composition comprising a hydrophobic fraction of embryogenic AGP effective for fostering somatic embryogenic competence; and
c) regenerating a second plant from said plant cell or tissue of step (b).

12. The method of claim 11 comprising, prior to step (b), the step of transforming said plant cell or tissue whereby a transformed plant is regenerated.

13. The method of claim 11 wherein the plant is cotton.

14. The method of claim 13 wherein the cotton plant is a variety selected from the group consisting of Upland cotton, Pima cotton, Egyptian cotton, Sea Island cotton, G. hirsutum, G. barbadense, tree cotton, Creole cotton, Levant cotton, Sturt's desert rose cotton, Thurber's cotton, and Hawaii cotton.

15. The method of claim 13 wherein the cotton plant is of an elite cotton line.

16. The method of claim 13 wherein the AGP composition comprises an embryogenic AGP of a cotton variety.

17. The method of claim 16 wherein the AGP composition effective for fostering somatic embryogenesis comprises pro-embryogenic AGP selected from the group consisting of de-glycosylated and de-arabinosylated AGP.

18. The method of claim 17 wherein the pro-embryogenic AGP is a protein having the amino acid sequence of SEQ ID NO: 25 or SEQ ID NO: 26.

19. The method of claim 17 wherein the pro-embryogenic AGP is a thrombin digest of the protein of SEQ ID No: 25.

20. The method of claim 17 wherein the pro-embryogenic AGP is a thrombin digest of the protein of SEQ ID No: 26.

21. A pro-embryogenic AGP composition comprising a hydrophobic fraction of embryogenic AGP of cotton.

22. The composition of claim 21 wherein the AGP is de-glycosylated or de-arabinosylated.

23. A pro-embryogenic AGP composition comprising a protein comprising a phytocyanin-like domain of an embryogenic AGP.

24. A pro-embryogenic AGP composition according to claim 23 comprising a protein having the amino acid sequence of SEQ ID NO: 25.

25. The composition of claim 23 comprising a thrombin digest of the protein of SEQ ID No: 25.

26. A pro-embryogenic AGP composition according to claim 23 comprising a protein having the amino acid sequence of SEQ ID NO: 26.

27. The composition of claim 23 comprising a thrombin digest of the protein of SEQ ID No: 25.

28. A method for making an AGP composition useful for fostering somatic embryogenic competence comprising:
a) providing embryogenic callus; and
b) harvesting AGP from said embryogenic callus and fractioning the AGP into hydrophilic and hydrophobic fractions and retaining the hydrophobic fraction.

29. A method of making pro-embryogenic AGP by expressing a protein comprising a phytocyanin-like domain of an embryogenic AGP.

30. The method of claim 29 wherein the protein has the amino acid sequence of SEQ ID No: 25 or SEQ ID NO:26.

31. (canceled)

32. The method of claim 30 comprising the added step of contacting the expressed protein with thrombin.
